(19) United States
(12) Patent Application Publication
(10) Pub. No.: US 2012/0190668 A1
(43) Pub. Date: Jul. 26, 2012

(54) ENHANCEMENT OF CELLULAR TRANSPLANTATION USING SMALL MOLEUCLE MODULATORS OF HEPATOCYTE GROWTH FACTOR (SCATTER FACTOR) ACTIVITY

(76) Inventors: Lambertus J.W.M. Oehlen, Westbury, NY (US); Itzhak D. Goldberg, Englewood, NJ (US)

(21) Appl. No.: 13/384,760

(22) PCT Filed: Aug. 12, 2010
(86) PCT No.: PCT/US2010/002232

§ 371 Date: Apr. 9, 2012

Related U.S. Application Data
(60) Provisional application No. 61/274,041, filed on Aug. 12, 2009.

(51) Int. Cl. A61K 31/55; A61K 31/4155; A61K 31/415; A61K 31/5377; A61K 31/4439; A61P 43/00; A61K 31/502; A61K 31/541; C12N 5/077; C12N 5/0793; C12N 5/078; C12N 5/071; A61K 31/454

(52) U.S. Cl. 514/217.05; 435/375; 514/406; 514/236.5; 514/341; 514/326; 514/248; 514/228.2

(57) ABSTRACT

The present invention provides methods for enhancing cellular transplantation by exposing cells in vitro or ex vivo, or a recipient of cells, to a small molecule hepatocyte growth factor/scatter factor mimetic. Exemplary compounds are described.

### BACKGROUND OF THE INVENTION

Various forms of cellular transplantation are being used to treat a number of diseases, and the future holds promise for expanding the types of cells and the target diseases that can be successfully treated or where function can be restored when the patient's organ or organ function has degenerated or failed. For example, islet cell transplants are used to treat diabetes; hepatocyte transplants are used to treat various liver diseases; stem cell transplantation is used for a variety of diseases; and cardiac cell transplantation in heart disease. Implantation of cells into the brain has promise for various CNS diseases. There is potential to transplant essentially any cell type from a donor, or cells from primary cell lines even including banked cell lines into a recipient for therapeutic benefit. Research is overcoming numerous challenges to the cellular transplantation field, for example rejection.

However, the cells used for such therapies are themselves vulnerable at every step in the procedure, from the harvesting, cultivation, isolation, purification, storage, and transplantation or implantation processes, as well as the cells becoming established in the recipient in order to restore function. Methods are needed to maintain survival and function of such cells during these steps to increase the success rate of cellular therapy, reduce the amount of tissue needed to prepare cells for transplant, and to assure that the cells reach their target and can become established and proliferate. By increasing cellular survival rate and increasing proliferation, the limited tissue available from cadaveric and other donors and from primary cell lines or even from cell cultures can be maximized.

Scatter factor (SF; also known as hepatocyte growth factor [HGF], and hereinafter referred to and abbreviated as HGF/SF) is a pleiotropic growth factor that stimulates cell growth, cell motility, morphogenesis and angiogenesis. HGF/SF is produced as an inactive monomer (−400 kDa) which is proteolytically converted to its active form. Active HGF/SF is a heparin-binding heterodimeric protein composed of a 62 kDa a chain and a 34 kDa β$ chain. HGF/SF is a potent mitogen for parenchymal liver, epithelial and endothelial cells (Matsumoto, K, and Nakamura, T., 1997, Hepatocyte growth factor (HGF) as a tissue organizer for organogenesis and regeneration. Biochem. Biophys. Res. Commun. 239, 639-44; Boros, P. and Miller, C. M., 1995, Hepatocyte growth factor: a multifunctional cytokine. Lancet 345, 293-5). It stimulates the growth of endothelial cells and also acts as a survival factor against endothelial cell death (Morishita, R, Nakamura, S, Nakamura, Y, Aoki, M, Moriguchi, A, Kida, I, Yo, Y, Matsumoto, K, Nakamura, T, Higaki, J, Ogihara, T, 1997, Potential role of an endothelium-specific growth factor, hepatocyte growth factor, on endothelial damage in diabetes. Diabetes 46:138-42). HGF/SF synthesized and secreted by vascular smooth muscle cells stimulates endothelial cells to proliferate, migrate and differentiate into capillary-like tubes in vitro (Grant, D. S, Kleinman, H. K., Goldberg, I. D., Bhargava, M. M., Nickoloff, B. J., Kinsella, J. L., Polyerini, P., Rosen, E. M., 1993, Scatter factor induces blood vessel formation in vivo. Proc. Natl. Acad. Sci. USA 90:1937-41; Morishita, R., Nakamura, S., Hayashi, S., Taniyama, Y., Moriguchi, A., Nagano, T., Taiji, M., Noguchi, H., Takeshita, S., Matsumoto, K., Nakamura, T., Higaki, J., Ogihara, T., 1999, Therapeutic angiogenesis induced by human recombinant hepatocyte growth factor in rabbit hind limb ischemia model as cytokine supplement therapy. Hypertension 33:1379-84). HGF/SF-containing implants in mouse subcutaneous tissue and rat cornea induce growth of new blood vessels from surrounding tissue. HGF/SF protein is expressed at sites of neovascularization including in tumors (Jeffers, M., Rong, S., Woude, G. F., 1996, Hepatocyte growth factor/scatter factor-Met signaling in tumorigenicity and invasion/metastasis. J. Mol. Med. 74:505-13; Moriyama, T., Kataoka, H., Koono, M., Wakisaka, S., 1999, Expression of hepatocyte growth factor/scatter factor and its receptor c-met in brain tumors: evidence for a role in progression of astrocytic tumors Int. J. Mol. Med. 3:531-6). These findings suggest that HGF/SF plays a significant role in the formation and repair of blood vessels under physiologic and pathologic conditions. Further discussion of angiogenic proteins may be found in U.S. Pat. Nos. 6,011,009 and 5,997,868, both of which are incorporated herein by reference in their entireties.

It is toward enhancing cellular transplantation during one or more of the in vitro, ex vivo or in vivo steps involving cells destined for transplantation that the present invention is directed.

### SUMMARY OF THE INVENTION

In one embodiment, methods for enhancing cellular transplantation are provided by exposing, in vitro, in vivo or ex vivo, cells for transplantation to compounds that mimic the biological activity of hepatocyte growth factor/scatter factor (HGF/SF). Such compounds, small molecule mimetics of HGF/SF, can be included in solutions or medium that cells harvested from a donor or culture, or in which such cells are maintained, transported, stored, cultured or administered. Such compounds can also be administered to the recipient of the cellular transplantation to enhance survival and proliferation of the cells in vivo. In another embodiment, compounds described herein reduce apoptosis of cells in vitro, ex vivo or in vivo. In another embodiment, compounds described herein increase survival of cells in vitro, ex vivo or in vivo by reducing apoptosis. In another embodiment, compounds described herein increase proliferation of cells in vitro, ex vivo or in vivo by reducing apoptosis. In another embodiment, administration of such a compound enhances angiogenesis and thereby improves engraftment of the transplanted cells. In other embodiments, compounds of the invention can be adminstered to the donor before harvest of the cells, or the tissues or organs from which cells will be isolated for transplantation. Such exposing can be to or during any one, any number of steps including but not limited to every step in the transplantation procedure. In another embodiment, such exposing is to an isolated tissue or organ before or during the harvesting of cells therefrom. Isolated can refer to a tissue or organ removed from the body, or a tissue or organ whose circulation or at least one anatomical connection to the body is disconnected during the harvesting.

The method is amenable to any of the numerous types of cells that can be transplanted for therapeutic benefit, such as but not limited to hepatocytes, neuronal cells, myocardial cells, and pancreatic cells including islet cells. In one embodiment, the cells express c-met, the receptor for HGF/SF.

Non-limiting examples of such compounds that are useful for the purposes herein have the structure:

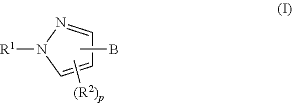

wherein p, R¹, R² and B are as described generally and in classes and subclasses herein

In certain embodiments, the present invention provides methods of using compounds of general formula (II^{A1}) and (III^{D1}),

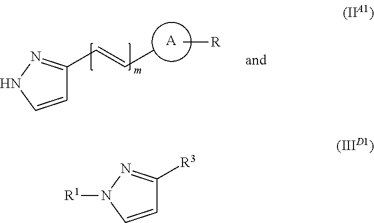

tautomers thereof, C(5)-positional isomers thereof; and pharmaceutical compositions thereof, as described generally and in subclasses herein.

In yet a further embodiment, the invention is also directed to methods for use of any of the purposes described herein compounds or pharmaceutical compositions comprising compounds with the general formulae XV and XVII:

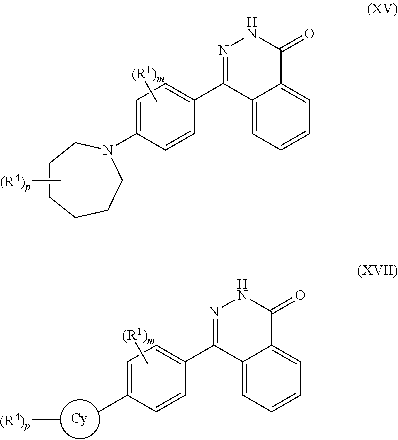

wherein the substituents depicted therein are described in detail below.

In another aspect, the invention provides methods for enhancing cellular transplantation for the treating or lessening the severity of a disease or condition such as but not limited to diabetes, liver disease, cerebrovascular disease, and cardiovascular disease. Other such diseases include liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency); damaged and/or ischemic organs; ischemia/reperfusion injury; stroke; cerebrovascular disease; myocardial ischemia; atherosclerosis; renal failure; renal fibrosis or idiopathic pulmonary fibrosis.

### DEFINITIONS

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, or alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. “Lower alkenyl” and “lower alkynyl” respectively include corresponding 1-6 carbon moieties.

In certain embodiments, the alkyl, alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, alkyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, butadienyl, dimethylbutadienyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is aliphatic or alicyclic, as defined herein. The term “aminoalkyl” refers to a group having the structure NH₂R′—, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments, the term “heteroaromatic moiety” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or more aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or more ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; C₁; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(—O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; C₁; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; C₁; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(—O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(R_{c})CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a heteroalicyclic group, which is a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. In certain embodiments, the term “heterocycloalkyl”, “heterocycle” or “heterocyclic” refers to a aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen. Representative heterocycles include, but are not limited to, heterocycles such as furanyl, thiofuranyl, pyranyl, pyrrolyl, pyrazolyl, imidazolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(ROC(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “halo alkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z})amine, where R., R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₂₋₆alkenylidene”, as used herein, refers to a substituted or unsubstituted, linear or branched unsaturated divalent radical consisting solely of carbon and hydrogen atoms, having from two to six carbon atoms, having a free valence “—” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation, particularly on the 1 position of the 3(5)-monosubstituted pyrazole derivatives of the invention. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. For example, compounds of formula II (and more generally, compounds of formula I where R¹ is hydrogen), can exist as a tautomer as shown below:

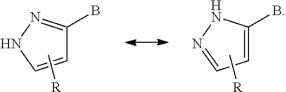

Thus, the present invention encompasses the 3-monosubstituted pyrazole compounds described herein (e.g., compounds of formula I, II, and related formulae II^{A}, II^{B}, II^{C}, etc. . . . ), as well as their tautomeric 5-monosubstituted pyrazole counterparts. Likewise, any compound shown as 5-monosubstituted pyrazole embraces its corresponding 3-monosubstituted tautomer.

The term “C(5)-positional isomer” as used herein refers to 1,5-disubstituted counterparts of the 1,3-disubstituted pyrazole compounds described herein. For example, the invention encompasses compounds of the formula (III^{B}) and its C(5)-positional isomer (III^{B′}):

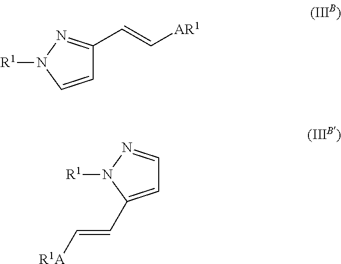

Thus, whether or not explicitly specified, the present invention encompasses the 1,3-disubstituted pyrazole compounds described herein (e.g., compounds of formula I, III, and related formulae III^{A}, III^{B}, III^{C}, III^{D}, etc. . . . ), as well as their C(5)-positional pyrazole counterparts. Likewise, any compound shown as 1,5-disubstituted pyrazole embraces its corresponding 1,3-disubstituted positional isomer.

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “isolated” when applied to the compounds of the present invention, refers to such compounds that are (i) separated from at least some components with which they are associated in nature or when they are made and/or (ii) produced, prepared or manufactured by the hand of man.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### DETAILED DESCRIPTION OF CERTAIN EMBODIMENTS OF THE INVENTION

The present invention is directed to the use of small molecule HGF/SF mimetic compounds described herein to enhance cellular transplantation therapy by, among other benefits, improving the survival, preserving the phenotype or enhancing or stimulating proliferation of cells intended for transplant, from the harvesting, isolation or cultivation phase of the process to implantation and post-engrafting survival and function. Compounds of the invention can be included in cellular cultivation and preservation fluids and medium, as well as administered to the cellular transplant recipient. Such in vitro and ex vivo uses can include compounds in any one or more of such steps. Exposing cells to one or more compounds of the invention enhances survival, preserves phenotype, stimulates proliferation, or any combination thereof. In other embodiments, compounds described herein can be administered to the donor before harvesting of cells or tissues or organs from which cells for transplantation will be isolated. In other embodiments the recipient of cellular transplantation therapy can be administered a compound of the invention to enhance the survival, engraftment, or proliferation of the cellular therapy. In another embodiment, such exposing can be to or during any one, any number of steps including but not limited to every step in the transplantation procedure. In another embodiment, such exposing is to an isolated tissue or organ before or during the harvesting of cells therefrom. Isolated can refer to a tissue or organ removed from the body, or a tissue or organ whose circulation or at least one anatomical connection to the body is disconnected during the harvesting. In another embodiment, the compounds described herein enhance angiogenesis in the recipient, promoting transplant engraftment and survival. In another embodiment, the compounds reduce apoptosis in the cells.

In another aspect, the invention provides methods for enhancing cellular transplantation for the treating or lessening the severity of a disease or condition such as but not limited to diabetes, liver disease, cerebrovascular disease, and cardiovascular disease. Such diseases include cirrhosis, liver fibrosis associated with hepatitis C, hepatitis B, delta hepatitis, chronic alcoholism, non-alcoholic steatohepatitis, extrahepatic obstructions (stones in the bile duct), cholangiopathies (primary biliary cirrhosis and sclerosing cholangitis), autoimmune liver disease, and inherited metabolic disorders (Wilson's disease, hemochromatosis, and alpha-1 antitrypsin deficiency). Other diseases include Parkinson's disease, stroke, myocardial infarctionrenal failure, renal fibrosis and idiopathic pulmonary fibrosis. These are merely illustrative and not meant to be limiting. The skilled artisan is well aware of the plethora of cells types that have been or can be transplated or where clinical trials are under way or promise of benefit has been identified; the methods embodied here are intended to be applicable to all such transplant procedures. Further non-limiting examples include bone marrow and bone marrow derived cells, lyphocytes and other immune cells or leukocytes (white blood cells) or leukocyte populations, stem cells including embryonic stem cells and mesenchymal stem cells, and myoblasts. Steps in the processing of such cells including harvesting, isolating, purifying, separating, expanding in vitro, and holding in vitro can subject the cells to adverse conditions reducing viability and usefulness. In another aspect, for these and any other cell types, the methods described here can reduce the amount of tissue required, or cells therefrom, for which a successful cellular transplant can be achieved.

The methods herein can also be used for enhancing survival, preserving the phenotype or enhancing or stimulating proliferation of any type of cells to be implanted into a body, including cells obtained from cell lines, cell cultures, cells isolated from tissues maintains in culture, and the like, including primary cell lines, during the manipulation, cultivation, in vitro treatment, growth or expansion phase in vitro before implantation. Compounds of the invention protect cells being handled in vitro or ex vivo from trauma, hypoxia, and other deleterious conditions that could induce cell dysfunction or death, including by apoptosis.

For example, the potential benefits of hepatocyte transplant are described in Fisher and Strom, Human Hepatocyte Transplantation: Worldwide Results, Transplantation 2006; 82: 441-449, and in Strom et al., Hepatocyte Transplantation: Clinical Experience and Potential for Future Use, Cell Transplantation, Vol. 15, Supplement 1, pp. S105-S110.

Cells for such therapies can be any one of a number of cell types of particular utility when transplanted. Cells can be obtained from a living or cadaveric donor, cell lines, primary cell cultures, by way of non-limiting examples are merely illustrative of the scope of the invention.

In another embodiment, the cells for transplantation express c-met, the receptor for HGF/SF.

### 1) General Description of Compounds of the Invention for the Uses Herein

In certain embodiments, compounds of the invention include compounds of the general formula (I) as further defined below:

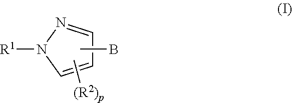

and tautomers and C(5)-positional isomers thereof;

wherein B is a C(3)- or C(5)-substituent selected from the group consisting of -AL¹-A, aryl, heteroaryl and heterocyclic; wherein AL¹ is an optionally substituted C₂₋₆ alkenyl moiety, and A is an optionally substituted alicyclic, heteroalicyclic, aromatic or heteroaromatic moiety;

R¹ is hydrogen, —C(═O)(CH₂)ₘR^{1A}, —C(═O)OR^{1A}, —C(═O)N(R^{1A})₂ or —SO₂R^{1A}; wherein m is an integer from 0-3; each occurrence of R^{1A} is independently hydrogen or an optionally substituted aliphatic, alicyclic, heteroaliphatic, heteroalicyclic, aromatic or heteroaromatic moiety;

p is one or more; and

each R² is independently selected from the group consisting of hydrogen, halogen, hydroxyl, —NO₂, —CN, an optionally substituted aliphatic, heteroaliphatic, alicyclic, heteroalicyclic, aromatic, heteroaromatic moiety; —OR^{R}, —S(═O). R^{d}, —NR^{b}R^{c}, and —C(═O)R^{a}; wherein n is 0-2, R^{R} is an optionally substituted aliphatic, heteroaliphatic, aromatic or heteroaromatic moiety;

R^{a}, for each occurrence, is independently selected from the group consisting of hydrogen, hydroxy, aliphatic, heteroaliphatic, aryl and heteroaryl;

R^{b} and R^{c}, for each occurrence, are independently selected from the group consisting of hydrogen; hydroxy; SO₂R^{d}; aliphatic, heteroaliphatic, aryl and heteroaryl;

R^{d}, for each occurrence, is independently selected from the group consisting of hydrogen; —N(R^{e})₂; aliphatic, aryl and heteroaryl; and

R^{e}, for each occurrence, is independently hydrogen or aliphatic.

In certain embodiments, the present invention defines particular classes of compounds which are of special interest. For example, one class of compounds of special interest includes those compounds of formula (I) wherein the nitrogen atom at position 1 is unsubstituted and the compound has the structure (II):

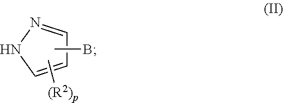

and tautomers thereof;

wherein p, R² and B are as defined generally above and in classes and subclasses herein.

Another class of compounds of special interest includes those compounds of formula (II) having the structure (II^{A}):

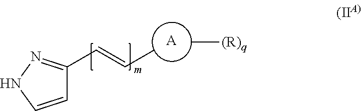

and tautomers thereof;

wherein A is an optionally substituted alicyclic, heteroalicyclic, aryl or heteroaryl moiety; m is an integer from 0-3; q is one or more, and each R is independently selected from the group consisting of hydrogen, halogen, hydroxyl, —NO₂, —CN, an optionally substituted aliphatic, heteroaliphatic, alicyclic, heteroalicyclic, aromatic, heteroaromatic moiety; —OR^{R}, —S(═O)ₙR^{d}, —NR^{b}R^{c}, and —C(═O)R^{a}; wherein n is 0-2, R^{R} is an optionally substituted aliphatic, heteroaliphatic, aromatic or heteroaromatic moiety;

R^{a}, for each occurrence, is independently selected from the group consisting of hydrogen, hydroxy, aliphatic, heteroaliphatic, aryl and heteroaryl;

R^{b} and R^{c}, for each occurrence, are independently selected from the group consisting of hydrogen; hydroxy; SO₂R^{d}; aliphatic, heteroaliphatic, aryl and heteroaryl;

R^{d}, for each occurrence, is independently selected from the group consisting of hydrogen; —N(R^{e})₂; aliphatic, aryl and heteroaryl; and

R^{e}, for each occurrence, is independently hydrogen or aliphatic.

In the foregoing formula, the [C═C]ₘ bond(s) can be the cis or the trans isomer.

Another class of compounds of special interest includes those compounds of formula (II) having the structure (II^{B}):

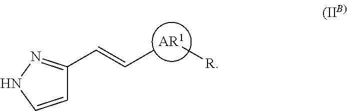

and tautomers thereof;

wherein R is as defined generally above and in classes and subclasses herein; and AR¹ is an optionally substituted aryl moiety. In the foregoing formula, the C═C bond can be the cis or the trans isomer.

Another class of compounds of special interest includes those compounds of formula (II) having the structure (II^{c}):

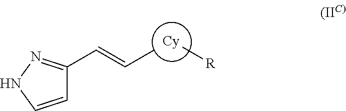

and tautomers thereof;

wherein R is as defined generally above and in classes and subclasses herein; and Cy is an optionally substituted heterocyclic moiety. In the foregoing formula, the C═C bond can be the cis or the trans isomer.

Another class of compounds of special interest includes those compounds of formula (I) wherein the nitrogen atom at position bears a substituent R¹ and the compound has the structure (III):

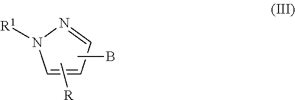

and C(5)-positional isomers thereof;

wherein B is as defined generally above and in classes and subclasses herein; and R¹ is —C(═O)(CH₂)ₘR^{1A}, —C(═O)OR^{1A}, —C(═O)N(R^{1A})₂ or —SO₂R^{1A}; wherein m is an integer from 0-3; and each occurrence of R^{1A} is independently hydrogen or an optionally substituted aliphatic, alicyclic, heteroaliphatic, heteroalicyclic, aromatic or heteroaromatic moiety.

Another class of compounds of special interest includes those compounds of formula (III) having the structure (III^{A}):

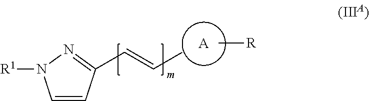

and C(5)-positional isomers thereof;

wherein R¹, R and A are as defined generally above and in classes and subclasses herein; and m is an integer from 0-3. In the foregoing formula, the [C═C]m bond(s) can be the cis or the trans isomer.

Another class of compounds of special interest includes those compounds of formula (III) having the structure (III^{B}):

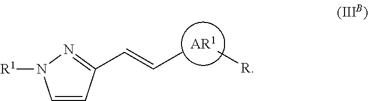

and C(5)-positional isomer thereof;

wherein R and R¹ are as defined generally above and in classes and subclasses herein; and AR¹ is an optionally substituted aryl moiety. In the foregoing formula, the C═C bond(s) can be the cis or the trans isomer.

Another class of compounds of special interest includes those compounds of formula (III) having the structure (III^{c}):

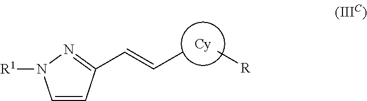

and C(5)-positional isomers thereof;

wherein R and R¹ are as defined generally above and in classes and subclasses herein; and Cy is an optionally substituted heterocyclic moiety. In the foregoing formula, the C═C bond(s) can be the cis or the trans isomer.

Another class of compounds of special interest includes those compounds of formula (III) having the structure (H^{1D}):

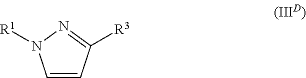

and C(5)-positional isomers thereof;

wherein R¹ is —SO₂R^{1A}; —C(═O)(CH₂)ₘR^{1A}, —C(═O)OR^{1A} or —C(═O)NHR^{1A}, wherein m is an integer from 0-3; and each occurrence of R^{1A} is independently an optionally substituted aliphatic, alicyclic, heteroaliphatic, aryl or heterocyclic moiety; and

R³ is a cis or trans —CH═CH-AR¹, —CH═CH—Cy, phenoxyphenyl, or a heterocyclic group; wherein AR¹ is an optionally substituted aryl moiety and Cy is an optionally substituted heterocyclic moiety.

In certain exemplary embodiments, when R¹ is —SO₂R^{1A}; —C(═O)R^{1A} or —C(═O)NHR^{1A}; wherein R^{1A} is alkyl or aryl; then R³ is not an optionally substituted cis or trans —CH═CH-heterocyclic, phenoxyphenyl, or a heterocyclic group.

A number of important subclasses of each of the foregoing classes deserve separate mention; these subclasses include subclasses of the foregoing classes in which:
i) R¹ is hydrogen;
ii) R¹ is —C(═O)R^{1A}, —C(═O)NHR^{1A} or —SO₂R^{1A}; wherein each occurrence of R^{1A} is independently alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety;
iii) R¹ is —C(═O)R^{1A}, —C(═O)NHR^{1A} or —SO₂R^{1A}; wherein each occurrence of R^{1A} is independently an alkyl, cycloalkyl, heterocyclic or aryl moiety;

iv) R¹ is —SO₂R^{1A}, —C(═O)(CH₂)ₘR^{1A}, —C(═O)OR^{1A} or —C(═O)NHR^{1A}, wherein m is an integer from 0-3; and each occurrence of R^{1A} is independently alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety;
v) R¹ is —SO₂R^{1A}, —C(═O)(CH₂)ₘR^{1A}, —C(═O)OR^{1A} or —C(═O)NHR^{1A}, wherein m is an integer from 0-3; and each occurrence of R^{1A} is independently an alkyl, cycloalkyl, heterocyclic or aryl moiety;
vi) R¹ is SO₂AL¹, C(═O)(CH₂)ₘAL¹, C(═O)OAL¹, C(═O)NHAL¹, SO₂Aryl, C(═O)(CH₂)ₘAryl, C(═O)OAryl, C(═O)OHeterocyclic, C(═O)(CH₂)ₘHeterocyclic, or C(═O)NHAryl; wherein m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;
vii) compounds of subset vi) above wherein AL¹ is alkyl or cycloalkyl;
viii) R¹ is C(═O)(CH₂)ₘAL¹; C(═O)(CH₂)ₘAryl or C(═O)Heterocyclic; wherein m⁻¹-3; AL¹ is an aliphatic or alicyclic moiety; and AL¹, the aryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₘR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{a})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;
ix) compounds of subset vii) above where AL¹ is alkyl or cycloalkyl;
x) R¹ is C(═O)O-AL¹ or C(═O)O-Aryl; wherein AL¹ is an aliphatic or alicyclic moiety; and AL¹ and the aryl moiety are optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{a})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
xi) compounds of subset x) above where AL¹ is alkyl or cycloalkyl;
xii) R¹ is SO₂AL′, C(═O)(CH₂)ₘAL¹, C(═O)NHAL¹, SO₂Aryl, C(═O)(CH₂)ₘAryl, C(═O)(CH₂)ₘHeterocyclic or C(═O)NHAryl; wherein m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;
xiii) compounds of subset xii) above where AL¹ is alkyl or cycloalkyl;
xiv) W is C(═O)(CH₂)ₘAL¹ wherein m is 1-3, C(═O)(CH₂)ₘAryl, C(═O)(CH₂)ₘHeterocyclic where m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;
xv) compounds of subset xiv) above where AL¹ is alkyl or cycloalkyl;
xvii) R¹ as SO₂AL¹, C(═O)AL¹, C(═O)NHAL¹, SO₂Aryl, C(═O)Aryl, or C(═O)NHAryl, wherein AL¹ is an aliphatic or alicyclic moiety; and AL¹ and the aryl moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;
xviii) compounds of subset xvii) above wherein AL¹ is alkyl or cycloalkyl;
xix) R¹ is C(═O)Aryl optionally substituted with one or more substituents independently selected from the group consisting of hydrogen; CN; carboxy ester; —C(═O)R^{a}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl substituted with one or more substituents independently selected from halogen, hydroxy, C₁₋₅ alkoxy, nitro, and N(R^{e})₂, or C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;
xx) B or R³ is a cis or trans CHCHAryl, CHCHHeterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;
xxi) B or R³ is a cis or trans CHCHAryl, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;
xxii) B or R³ is a cis or trans CHCHheterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅alkoxy, nitro, and N(R^{e})₂;
xxiii) R is one or more substituents selected from the group consisting of hydrogen, halogen, hydroxyl, —NO₂, —CN, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety; hydrogen, alkyl, heteroalkyl, aryl, heteroaryl, -(alkyl)aryl, -(alkyl)heteroaryl, —OR^{R}, —S(═O)ₙR^{R}, —N(R^{R}), —SO₂N(R^{R})₂, —C(═O)R^{R}, —C(═O)N(R^{R})₂, —C(═O)OR^{R}, —N(R^{R})C(═O)R^{R} or —N(R^{R})SO₂R^{R}; wherein n is 0-2, and R^{R}, for each occurrence, is independently hydrogen, lower alkyl, lower heteroalkyl, aryl, heteroaryl, -(alkyl)aryl, or -(alkyl)heteroaryl;
xxiv) R is one or more substituents selected from the group consisting of hydrogen, halogen, hydroxyl, —NO₂, —CN, alkoxy, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl, -(heteroalkyl)heteroaryl moiety, —S(═O)ₙR^{d}, —NR^{b}R^{c}, and —C(═O)R^{a}; wherein n is 0-2;
xxv) each R is independently selected from hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
xxvi) each R is independently selected from hydrogen; halogen; hydroxy; nitro; CN; C₁₋₆ alkyl; C₁₋₆ alkoxy; haloC₁₋₆ alkoxy; —C(═O)R^{a}; —C(═O)Oa^{r}; —OR^{a} and —NR^{a}R^{b}; wherein R^{a} and R^{b} are independently lower alkyl or any two adjacent R^{a} groups, or R^{a} and R^{b} groups, taken together, may form a heterocyclic moiety;
xxvii) each R is independently selected from hydrogen; halogen; hydroxy or nitro;
xxviii) R^{a}, for each occurrence, is independently selected from the group consisting of hydrogen, hydroxy, C₁₋₆ alkyl, C₁₋₆ alkoxy, aryl, heteroaryl, and NR^{b}R^{C}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
xxix) R^{b} and R^{c}, for each occurrence, are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
xxx) R^{d}, for each occurrence, is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;
xxxi) R^{c}, for each occurrence, is independently hydrogen or C₁₋₆ alkyl;
xxxii) R^{f} and R^{g}, for each occurrence, are independently selected from the group consisting of hydrogen; hydroxy; SO₂R^{d}; C₁₋₆ alkyl substituted with one or more substituents independently selected from halogen, hydroxy, C₁₋₅ alkoxy, nitro, and N(R^{e})₂; alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
xxxiii) R² is one or more substituents selected from the group consisting of hydrogen, halogen, hydroxyl, —NO₂, —CN, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety; hydrogen, alkyl, heteroalkyl, aryl, heteroaryl, -(alkyl)aryl, -(alkyl)heteroaryl, —OR^{R}, —S(═O)ₙR^{R}, —N(R^{R})₂, —SO₂N(R^{R})₂, —C(═O)R^{R}, —C(═O)N(R^{R})₂, —C(═O)OR^{R}, —N(R^{R})C(═O)R^{R} or —N(R^{R})SO₂R^{R}; wherein n is 0-2, and R^{R}, for each occurrence, is independently hydrogen, lower alkyl, lower heteroalkyl, aryl, heteroaryl, -(alkyl)aryl, or -(alkyl)heteroaryl;
xxxiv) R² is one or more substituents selected from the group consisting of hydrogen, halogen, hydroxyl, —NO₂, —CN, alkoxy, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl, -(heteroalkyl)heteroaryl moiety, —S(═O)ₙR^{d}, —NR^{b}R^{c}, and —C(═O)R^{a}; wherein n is 0-2;
xxxv) R² is one or more substituents selected from hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
xxxvi) R² is one or more substituents selected from hydrogen; halogen; hydroxy; nitro; CN; C₁₋₆ alkyl; C₁₋₆ alkoxy; haloC₁₋₆ alkoxy; —C(═O)R^{a}; —C(═O)OR^{a}; —OR^{a} and —NR^{a}R^{b}; wherein R^{a} and R^{b} are independently lower alkyl or any two adjacent R^{a} groups, or R^{a} and R^{b} groups, taken together, may form a heterocyclic moiety;
xxxvii) A is an alicyclic, heteroalicyclic, aromatic or heteroaromatic moiety;
xxxviii) A is an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S;
xxxix) A is an aromatic or non-aromatic 5-6 membered monocyclic ring or 8-12 membered bicyclic ring, optionally substituted with one or more substituents selected from hydrogen; halogen; hydroxy; nitro; CN; C₁₋₆ alkyl; C₁₋₆ alkoxy; haloC₁₋₆ alkoxy; —C(═O)R^{a}; —C(═O)OR^{a}; —OR^{a} and —NR^{a}R^{b}; wherein R^{a} and R^{b} are independently lower alkyl or any two adjacent R^{a} groups, or R^{a} and R^{b} groups, taken together, may form a heterocyclic moiety;
xl) A is an aromatic or non-aromatic 5-6 membered monocyclic ring or 8-12 membered bicyclic ring, optionally substituted with one or more substituents selected from hydrogen; Cl; hydroxy; nitro; CN; —OCF₃; —C(═O)Omen; —C(═O)Me; —OMe; methyldioxyl; —NMe₂ and morpholinyl;
xli) A is optionally substituted aryl;
xlii) A is optionally substituted phenyl or naphthyl;
xliii) A is optionally substituted heteroaryl;
xliv) A has the structure:

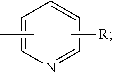

wherein R represents one or more substituents, as defined in subsets xxiii)-xxvii);
xlv) A is an optionally substituted C₁₋₆cycloalkyl or C₁₋₆cycloalkenyl moiety;
xlvi) A is optionally substituted cyclohexenyl;
xlvii) A is an optionally substituted heterocyclic moiety;
xlviii) A and/or Cy is one of:

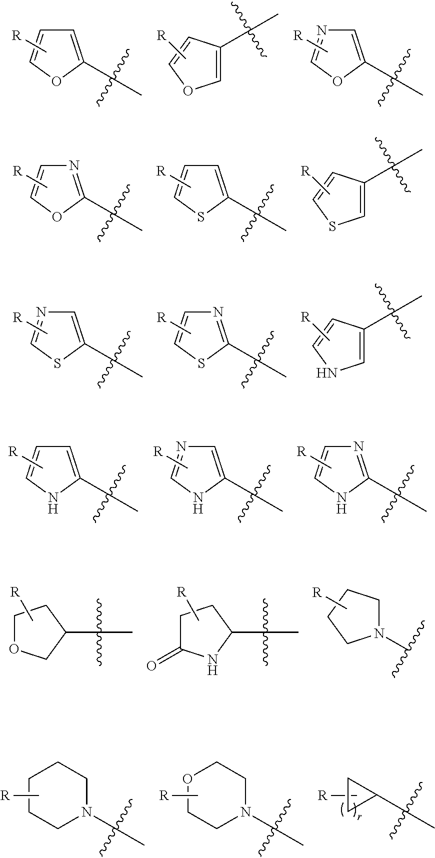

wherein R represents one or more substituents, as defined in subsets xxiii)-xxvii);

and r is an integer from 1-6;

xlix) A and/or Cy is an optionally substituted 5-membered heterocyclic moiety having the structure:

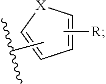

wherein R represents one or more substituents, as defined in subsets xxiii)-xxvii);
and X is O, S or NR^{N}; wherein R^{N} is hydrogen, lower alkyl, aryl, acyl or a nitrogen protecting group;

l) A and/or Cy is an optionally substituted 5-membered heterocyclic moiety having the structure:

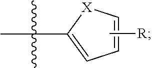

wherein R represents one or more substituents, as defined in subsets xxiii)-xxvii); and X is O, S or NR^{N}; wherein R^{N} is hydrogen, lower alkyl, aryl, acyl or a nitrogen protecting group;

li) B is a moiety having the structure:

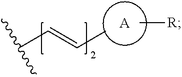

wherein A and R are as defined in classes and subclasses herein;

lii) B is a moiety having one of the structures:

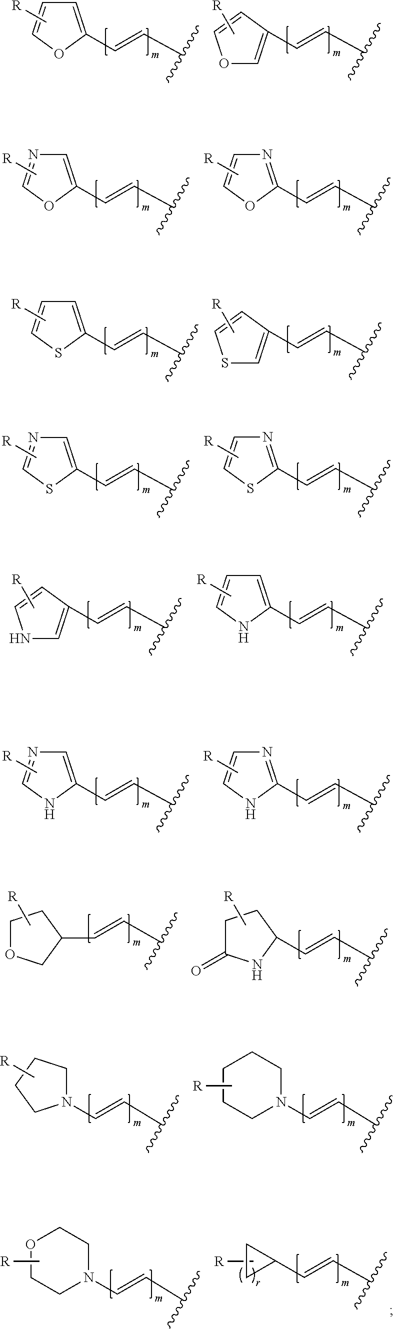

wherein R represents one or more substituents, as defined in subsets xxiii)-xxvii); m is an integer from 1-3; and r is an integer from 1-6; and wherein any of the [C═C]ₘ bond(s) can be the cis or the trans isomer;

liii) AR is phenyl or naphthyl;

liv) AR¹ is phenyl or naphthyl;

lv) p is 1;

lvi) p is 2;

lvii) p is 3;

lviii) q is 1;

lix) q is 2;

lx) q is 3;

lxi) q is 4;

lxii) q is 5;

lxiii) q is 6;

lxiv) m is 1;

lxv) m is 2;

lxvi) m is 3;

lxviii) m is 4;

lxix) m is 5; and/or

lxx) m is 6.

It will be appreciated that for each of the classes and subclasses described above and herein, any one or more occurrences of aliphatic and/or heteroaliphatic may independently be substituted or unsubstituted, linear or branched, saturated or unsaturated; any one or more occurrences of alicyclic and/or heteroalicyclic may independently be substituted or unsubstituted, saturated or unsaturated; and any one or more occurrences of aryl and/or heteroaryl may independently be substituted or unsubstituted.

The reader will also appreciate that all possible combinations of the variables described in i)-through lxx) above (e.g., R, R¹, and B, among others) are considered part of the invention. Thus, the invention encompasses any and all compounds of formula I generated by taking any possible permutation of variables R, R¹, and B, and other variables/substituents (e.g., A, R^{1A}, etc.) as further defined for R, R¹, and B, described in i)-through lxx) above.

For example, an exemplary combination of variables described in i)-through lxx) above includes those compounds of Formula I wherein:
B is a C(3)- or C(5)-substituent selected from the group consisting of optionally substituted cis or trans CHCHAryl, CHCHHeterocyclic, phenoxyphenyl and a heterocyclic group;
R¹ is C(═O)Aryl optionally substituted with one or more substituents independently selected from the group consisting of hydrogen; CN; carboxy ester; —C(═O)R^{a}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl substituted with one or more substituents independently selected from halogen, hydroxy, C₁₋₅ alkoxy, nitro, and N(R^{e})₂, or C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; and
each R is independently selected from hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂ᵢ
wherein R^{a}, for each occurrence, is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c}, for each occurrence, are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d}, for each occurrence, is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and R^{c}, for each occurrence, is independently hydrogen or C₁₋₆ alkyl.

Other exemplary combinations are illustrated by compounds of the following subgroups I-XII:

I. Compounds Having the Structure:

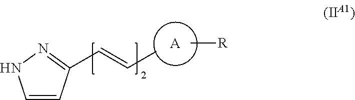

tautomers thereof; and pharmaceutically acceptable derivatives thereof;
wherein A and R are as defined generally and in classes and subclasses herein. In certain embodiments, A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In the foregoing formula, the [C═C]₂ bond(s) can be the cis or the trans isomer. In certain other embodiments, R is selected from the group consisting of hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein each occurrence of R^{a} is independently selected from the group consisting of hydrogen, hydroxy, C₁₋₆ alkyl, C₁₋₆ alkoxy, aryl, heteroaryl, and NR^{b}R^{C}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
each occurrence of R^{d} is independently selected from the group consisting of hydrogen; N(R^{e})₂; C₁₋₆ alkyl optionally substituted with one or more substituents independently selected from halogen, hydroxy, Cl_{—}5 alkoxy, nitro, and N(R^{e})₂; aryl and heteroaryl; and
each occurrence of R^{c} is independently hydrogen or C₁₋₆ alkyl.

A non-limiting example of compounds of this subgroup includes:

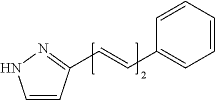

II. Compounds Having the Structure:

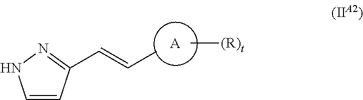

tautomers thereof, and pharmaceutically acceptable derivatives thereof;
wherein A and R are as defined generally and in classes and subclasses herein and t is 1-5. The C═C bond can be the cis or the trans isomer.

In certain exemplary embodiments, A is an aromatic or non-aromatic 5-6 membered monocyclic ring, optionally containing 1-4 heteroatoms selected from N, O or S; or an aromatic or non-aromatic 8-12 membered bicyclic ring, optionally containing 1-6 heteroatoms selected from N, O or S;
and each R is independently selected from the group consisting of hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
   wherein each occurrence of R^{a} is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
   each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
   each occurrence of R^{d} is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
   each occurrence of R^{c} is independently hydrogen or C₁₋₆ alkyl;

or a prodrug, salt, hydrate, or ester thereof.

III. Compounds Having the Structure:

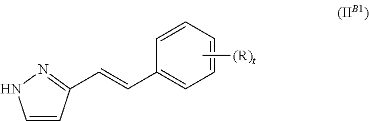

tautomers thereof, and pharmaceutically acceptable derivatives thereof;
wherein t and R are as defined generally and in classes and subclasses herein. In certain embodiments, R is as defined for subgroup II above. In the foregoing formula, the C═C bond can be the cis or the trans isomer.

Non-limiting examples of compounds this subgroup include:
4-(2-(1H-pyrazol-3-yl)vinyl)-N,N-dimethylaniline
3-(2,6-dichlorostyryl)-1H-pyrazole
3-(2-(benzo[d][1,3]dioxol-4-yl)vinyl)-1H-pyrazole
3-(2-(naphthalen-2-yl)vinyl)-1H-pyrazole
3-(2-(trifluoromethyl)styryl)-1H-pyrazole
3-(2-chloro-4-(trifluoromethyl)styryl)-1H-pyrazole
3-(4-(diethoxymethyl)styryl)-1H-pyrazole
3-(4-methoxystyryl)-1H-pyrazole and
3-styryl-1H-pyrazole.

IV. Compounds Having the Structure:

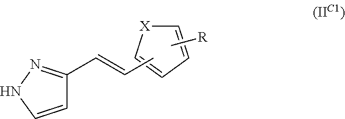

tautomers thereof, and pharmaceutically acceptable derivatives thereof;

wherein R is as defined generally and in classes and subclasses herein; and X is O, S or NR^{N} wherein R^{N} is hydrogen, alkyl, heteroalkyl, aryl, heteroaryl, -(alkyl)aryl, -(alkyl)heteroaryl, acyl or a nitrogen protecting group. In the foregoing formula, the C═C bond can be the cis or the trans isomer. In certain embodiments, R is as defined for subgroup II above.

Non-limiting examples of compounds of this subgroup include:
2-(2-(1H-pyrazol-3-yl)vinyl)-1H-indole
3-(2-(1H-pyrrol-2-yl)vinyl)-1H-pyrazole
3-(2-(1H-pyrrol-3-yl)vinyl)-1H-pyrazole
3-(2-(5-nitrofuran-2-yl)vinyl)-1H-pyrazole
3-(2-(furan-2-yl)vinyl)-1H-pyrazole
3-(2-(thiophen-2-yl)vinyl)-1H-pyrazole
3-(2-(thiophen-3-yl)vinyl)-1H-pyrazole and
3-(2-(furan-3-yl)vinyl)-1H-pyrazole.

V. Compounds Having the Structure:

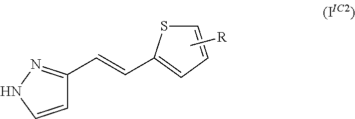

tautomers thereof, and pharmaceutically acceptable derivatives thereof;

wherein R is as defined generally and in classes and subclasses herein. In certain embodiments, R is as defined in subgroup II above. In the foregoing formula, the —C═C— bond can be the cis or the trans isomer.

VI. Compounds Having the Structure:

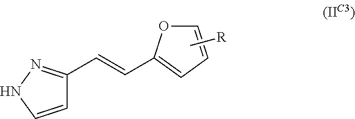

tautomers thereof, and pharmaceutically acceptable derivatives thereof;

wherein R is as defined generally and in classes and subclasses herein. In certain embodiments, R is as defined in subgroup II above. In the foregoing formula, the —C═C— bond can be the cis or the trans isomer.

VII. Compounds Having the Structure:

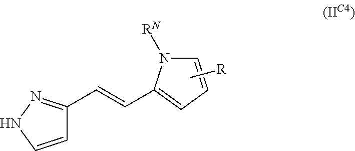

tautomers thereof, and pharmaceutically acceptable derivatives thereof;

wherein R is as defined generally and in classes and subclasses herein; and R^{N} is hydrogen, alkyl, heteroalkyl, aryl, heteroaryl, -(alkyl)aryl, -(alkyl)heteroaryl, acyl or a nitrogen protecting group. In certain embodiments, R is as defined in subgroup II above. In certain other embodiments, R^{N} is hydrogen. In the foregoing formula, the —C═C— bond can be the cis or the trans isomer.

In another broad aspect of the present invention, the following disubstituted compounds and their C(5)-positional isomers are embraced herein, such compounds exhibiting HGF/SF mimicking/modulating activity, and in particularly activity similar to that of HGF/SF.

VIII. Compounds Having the Structure:

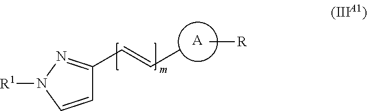

a. C(5)-positional isomers thereof; and pharmaceutically acceptable derivatives thereof;
wherein R¹ and R are as defined generally and in classes and subclasses herein; m is an integer from 0-3; and A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In the foregoing formula, the [C═C]ₘ bond(s) can be the cis or the trans isomer. In certain other embodiments, R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein AL² is an alkyl or cycloalkyl moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{a}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃.

In certain embodiments, R is as defined in subgroup II above.

IX. Compounds Having the Structure:

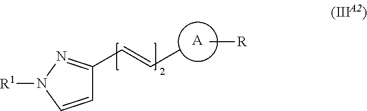

C(5)-positional isomers thereof; and pharmaceutically acceptable derivatives thereof;
wherein A, R¹ and R are as defined generally and in classes and subclasses herein. In the foregoing formula, the [C═C]₂ bonds can be the cis or the trans isomer. In certain embodiments, A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In certain other embodiments, R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 0-3; AL² is an alkyl or cycloalkyl moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; or COCH₂OC₂H₅OCH₃. In certain embodiments, R is as defined in subgroup II above.

X. Compounds Having the Structure:

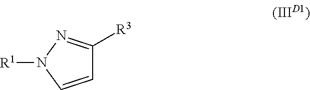

C(5)-positional isomers thereof; and pharmaceutically acceptable derivatives thereof;
wherein R¹ is C(═O)(CH₂)ₘAL², C(═O)OAL², C(═O)(CH₂)ₘAryl, C(═O)OAryl, C(═O)Heteroaryl or C(═O)Heterocyclic; where m is an integer from 1-3; AL² is an aliphatic or alicyclic moiety; and AL², the aryl, heteroaryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
R³ is a cis or trans CHCHAryl, CHCHHeterocyclic, phenoxyphenyl, or a heterocyclic group, wherein the aryl, heterocyclic or phenoxyphenyl moiety may be optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{a})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

In certain embodiments, for the compounds of formula (III^{D1}) above, AL² is an alkyl or cycloalkyl moiety.

In certain embodiments, for the compounds of formula (III^{D1}) above, R³ is a cis or trans CHCHHeterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{a})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{C}, wherein C₁₋₆ alkyl and C₁₋₆ alkoxy are optionally substituted with one or more substituents independently selected from halogen, hydroxy, C₁₋₅ alkoxy, nitro, and N(R^{a})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{a})₂; C₁₋₆ alkoxy optionally substituted with one or more substituents independently selected from halogen, hydroxy, C₁₋₅ alkoxy, nitro and N(R^{a})₂; aryl optionally substituted with one or more substituents independently selected from halogen, hydroxy, C₁₋₄ alkyl, C₁₋₅ alkoxy, nitro, and N(R^{a})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

Non-limiting examples of compounds of this subgroup include:
(1-methyl-1H-pyrrol-2-yl)(3-(2-(thiophen-2-yl)vinyl)-1H-pyrazol-1-yl)methanone
(3-(2-(furan-2-yl)vinyl)-1H-pyrazol-1-yl)(1-methyl-1H-pyrrol-2-yl)methanone
(3-(2-(furan-2-yl)vinyl)-1H-pyrazol-1-yl)(6-morpholinopyridin-3-yl)methanone
(3-(2-(furan-2-yl)vinyl)-1H-pyrazol-1-yl)(thiophen-2-yl)methanone
(3-(2-(furan-2-yl)vinyl)-1H-pyrazol-1-yl)(thiophen-3-yl)methanone
(3-(2-(thiophen-2-yl)vinyl)-1H-pyrazol-1-yl)(thiophen-3-yl)methanone
(5-chloro-4-methoxythiophen-3-yl)(3-(2-(thiophen-2-yl)vinyl)-1H-pyrazol-1-yl)methanone
(5-nitrothiophen-3-yl)(3-(2-(thiophen-2-yl)vinyl)-1H-pyrazol-1-yl)methanone
(6-chloropyridin-3-yl)(3-(2-(thiophen-2-yl)vinyl)-1H-pyrazol-1-yl)methanone
1-(4-(3-(2-(thiophen-2-yl)vinyl)-1H-pyrazole-1-carbonyl)piperidin-1-yl)ethanone
furan-2-yl(3-(2-(furan-2-yl)vinyl)-1H-pyrazol-1-yl)methanone
furan-2-yl(3-(2-(thiophen-2-yl)vinyl)-1H-pyrazol-1-yl)methanone
thiophen-2-yl(3-(2-(thiophen-2-yl)vinyl)-1H-pyrazol-1-yl)methanone and
3-(5-(3-chlorostyryl)-1H-pyrazol-1-yl)-5-methyl-4H-1,2,4-triazol-4-amine.

As mentioned above and herein throughout, although the compound structures depicted herein are substituted at the 1 and 3 positions, the invention embraces such positional isomers where the 3-substituent is at the 5 position, and any combination thereof.

In another aspect of compounds of Formula (III^{D1}), R³ is a cis or trans CHCHAryl, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, NR^{b}R^{c}C, —S(O)ₙR^{d} where n=0-2, hydroxy, C₁₋₆ alkoxy, haloC₁₋₆alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{C}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

Non-limiting examples of compounds of this subgroup include:
(3-(2,6-dichlorostyryl)-1H-pyrazol-1-yl)(thiophen-2-yl)methanone
(3-styryl-1H-pyrazol-1-yl)(thiophen-2-yl)methanone
(3-styryl-1H-pyrazol-1-yl)(thiophen-3-yl)methanone
(5-nitrothiophen-3-yl)(3-styryl-1H-pyrazol-1-yl)methanone
(6-morpholinopyridin-3-yl)(3-styryl-1H-pyrazol-1-yl)methanone and
furan-2-yl(3-styryl-1H-pyrazol-1-yl)methanone.

XI. Compounds Having the Structure:

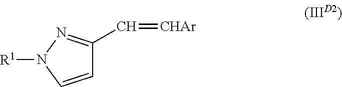

C(5)-positional isomers thereof; and pharmaceutically acceptable derivatives thereof;
wherein R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 1-3; AL² is an aliphatic or alicyclic moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
CHCHAr is a cis or trans CH═CHAryl optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

In certain embodiments, for compounds of Formula (III^{D2}), R¹ is C(═O)(CH₂)ₘAL², C(═O)OAL², C(═O)(CH₂)ₘAryl, C(═O)OAryl, C(═O)OHeterocyclic or

C(═O)(CH₂)ₘHeterocyclic; wherein m is an integer from 1-3; AL² is an aliphatic or alicyclic moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₘR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃.
In certain other embodiments, for compounds of Formula (III^{D2}), R¹ is SO₂AL², C(═O)AL², C(═O)NHAL², SO₂Aryl, C(═O)Aryl, or C(═O)NHAryl; wherein AL² is an aliphatic or alicyclic moiety; and AL² and the aryl moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃.

In certain embodiments, for the compounds of subgroup XI above, AL² is an alkyl or cycloalkyl moiety.

XII. Compounds Having the Structure:

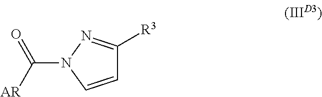

C(5)-positional isomer thereof; and pharmaceutically acceptable derivatives thereof;
wherein AR is an optionally fused 3-12 membered aromatic or alicyclic mono- or bicyclic-ring containing 0-3 heteroatoms selected from the group consisting of N, O, and S optionally substituted with one or more substituents independently selected from the group consisting of hydrogen; halogen; hydroxy; nitro; CN; aryl; heteroaryl; heterocycle; carboxy ester; —C(═O)R^{a}, —NR^{b}R^{c}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; and
R³ is a cis or trans CHCHheterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{C}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;
R^{e} is hydrogen or C₁₋₆ alkyl; and
R^{f} and R^{g} are independently selected from the group consisting of hydrogen; hydroxy; SO₂R^{d}; C₁₋₆ alkyl substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂.

In certain embodiments, when AR is aryl substituted with C₁₋₆alkyl, the C₁₋₆alkyl moiety is substituted. In certain exemplary embodiments, the substituents are independently selected from halogen, hydroxy, C₁₋₅ alkoxy, nitro and N(R^{e})₂.

It will be appreciated that each of the compounds described herein and each of the subclasses of compounds described above (I-XII) may be substituted as described generally herein, or may be substituted according to any one or more of the subclasses described above and herein [e.g., i)-lxx)].

In another embodiment, compounds useful for the purposes described herein have the structure of the general Formula (XV) as further defined below:

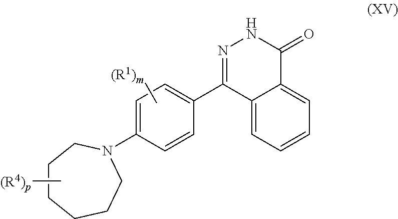

or pharmaceutically acceptable derivative thereof;
wherein m is an integer from 1 to 4;
p is an integer from 1 to 6;
each occurrence of R¹ and R⁴ is independently hydrogen, halogen, hydroxyl, —NO₂, —NH₂, —CN, an optionally substituted aliphatic, alicyclic, heteroaliphatic, heterocyclic, aromatic or heteroaromatic moiety, —OR^{R}, —S(═O)ₙR^{d}, —NR^{b}R^{c}, —C(═O)R^{a}, —OPO₂OR^{a} or —C(═O)OR^{a}; wherein n is 0-2, R^{R} is an optionally substituted aliphatic, alicyclic, heteroaliphatic, heterocyclic, aromatic, heteroaromatic or acyl moiety;
R^{a}, for each occurrence, is independently selected from the group consisting of hydrogen and an optionally substituted aliphatic, alicyclic, heteroaliphatic, heterocyclic, aromatic, or heteroaromatic moiety;
R^{b} and R^{c}, for each occurrence, are independently selected from the group consisting of hydrogen; hydroxy; SO₂R^{d}; and aliphatic, alicyclic, heteroaliphatic, heterocyclic, aromatic, heteroaromatic or acyl moiety;
R^{d}, for each occurrence, is independently selected from the group consisting of hydrogen; —N(R^{e})₂; aliphatic, alicyclic, heteroaliphatic, heterocyclic, aromatic or heteroaromatic; and
R^{c}, for each occurrence, is independently hydrogen or aliphatic.

In certain other embodiments, compounds of formula (XV) are defined as follows:
m is an integer from 1 to 4;
p is an integer from 1 to 6;
each occurrence of R¹ and R⁴ is independently hydrogen, halogen, hydroxyl, —NO₂, —NH₂, —CN, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a}, —OPO₂OR^{a} or —C(═O)Or^{a}; wherein n is 0-2, R^{R} is an optionally substituted alkyl, heteroalkyl, cycloalkyl, heterocyclic, aryl, heteroaryl or acyl moiety;
R^{a}, for each occurrence, is independently hydrogen or an optionally substituted alkyl, heteroalkyl, cycloalkyl, heterocyclic, aryl or heteroaryl moiety;
R^{b} and R^{c}, for each occurrence, are independently hydrogen, hydroxy, SO₂R^{d}, or an alkyl, heteroalkyl, cycloalkyl, heterocyclic, aryl, heteroaryl or acyl moiety;
R^{d}, for each occurrence, is independently hydrogen, —N(R^{e})₂, alkyl, heteroalkyl, cycloalkyl, heterocyclic, aryl or heteroaryl; and
R^{e}, for each occurrence, is independently hydrogen or alkyl.

In another aspect, the invention is directed to the use for the purposes described herein of the following compounds and pharmaceutical compositions comprising compounds of formula (XVI):

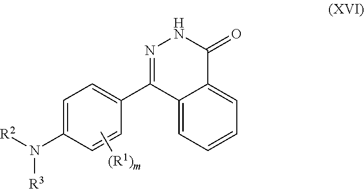

or pharmaceutically acceptable derivatives thereof;
m is an integer from 1 to 4;
each occurrence of R¹ is independently hydrogen, halogen, hydroxyl, —NO₂, —NH₂, —CN, an optionally substituted aliphatic, alicyclic, heteroaliphatic, heterocyclic, aromatic or heteroaromatic moiety, —OR^{R}, —S(═O)ₙR^{d}, —NR^{b}R^{c}, —C(═O)R^{a}, —OPO₂OR^{a} or —C(═O)OR^{a}; wherein n is 0-2, R^{R} is an optionally substituted aliphatic, alicyclic, heteroaliphatic, heterocyclic, aromatic, heteroaromatic or acyl moiety;
R² and R³ are independently hydrogen, hydroxyl, —NH₂, an optionally substituted aliphatic, heteroaliphatic, alicyclic, heterocyclic, aromatic or heteroaromatic moiety, —OR^{R}, —S(═O)ₙR^{d}, —NR^{b}R^{c}, —C(═O)R^{a} or —C(═O)OR^{a}; wherein n is 0-2, R^{R} is an optionally substituted aliphatic, heteroaliphatic, alicyclic, heterocyclic, aromatic or heteroaromatic or acyl moiety; or R² and R³ taken together with the nitrogen to which they are attached form an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{a}, for each occurrence, is hydrogen or an optionally substituted aliphatic, alicyclic, heteroaliphatic, heterocyclic, aromatic, or heteroaromatic moiety;

R^{b} and R^{c}, for each occurrence, are independently hydrogen, hydroxy, SO₂R^{d}, or an optionally substituted aliphatic, alicyclic, heteroaliphatic, heterocyclic, aromatic, heteroaromatic or acyl moiety;

R^{d}, for each occurrence, is independently hydrogen, —N(R^{e})₂, or an optionally substituted aliphatic, alicyclic, heteroaliphatic, heterocyclic, aromatic or heteroaromatic moiety; and

R^{e}, for each occurrence, is independently hydrogen or aliphatic.

In certain other embodiments, compounds of formula (XVI) are defined as follows:
m is an integer from 1 to 4;
each occurrence of R¹ is independently hydrogen, halogen, hydroxyl, —NO₂, —NH₂, —CN, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a}, —OPO₂OR^{a} or —C(═O)Or^{a}; wherein n is 0-2, R^{R} is an optionally substituted alkyl, heteroalkyl, cycloalkyl, heterocyclic, aryl, heteroaryl or acyl moiety;
R² and R³ are independently hydrogen, hydroxyl, —NH₂, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a} or —C(═O)OR^{a}; wherein n is 0-2, R^{R} is an optionally substituted alkyl, heteroalkyl, cycloalkyl, heterocyclic, aryl, heteroaryl or acyl moiety; or R² and R³ taken together with the nitrogen to which they are attached form a optionally substituted heteroaryl or heterocyclic group comprising 4-10 ring members and 0-3 additional heteroatoms selected from the group consisting of O, N and S; the heteroaryl or heterocyclic group optionally further substituted with one or more optionally substituted alkyl, cycloalkyl, heteroalkyl, heterocyclic, aryl, heteroaryl or acyl groups;
wherein R^{a}, for each occurrence, is independently hydrogen or an optionally substituted alkyl, heteroalkyl, cycloalkyl, heterocyclic, aryl or heteroaryl moiety;
R^{b} and R^{c}, for each occurrence, are independently hydrogen, hydroxy, SO₂R^{d}, or an alkyl, heteroalkyl, cycloalkyl, heterocyclic, aryl, heteroaryl or acyl moiety;
R^{d}, for each occurrence, is independently hydrogen, —N(R^{e})₂, alkyl, heteroalkyl, cycloalkyl, heterocyclic, aryl or heteroaryl; and
R^{e}, for each occurrence, is independently hydrogen or alkyl.

In certain embodiments, the present invention defines certain classes of compounds which are of special interest for the uses and methods described herein. For example, one class of compounds of special interest includes those compounds having the structure of formula (XVII) in which the compound has the structure:

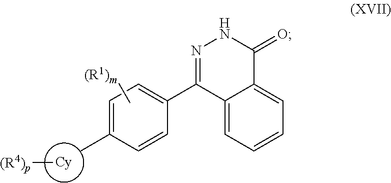

wherein m, p, R¹ and R⁴ are as defined in classes and subclasses herein; and Cy is an optionally substituted N-linked 5- to 10-membered heterocyclic group.

Another class of compounds of special interest includes those compounds having the structure of formula (XVIII) in which the compound has the structure:

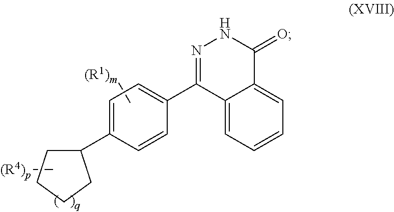

wherein m, p, R¹ and R⁴ are as defined in classes and subclasses herein; and q is an integer selected from 1, 2 or 4.

For the uses and methods described herein, a number of important subclasses of each of the foregoing classes of compounds of formulae (XV), (XVI), (XVII) and (XVIII) deserve separate mention; these subclasses include subclasses of the foregoing classes in which:
i. each occurrence of R¹ is independently hydrogen, halogen, hydroxyl, —NO₂, —NH₂, —CN, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a}, —OPO₂OR^{a} or —C(═O)OR^{a}; wherein n is 0-2, R^{R} is an optionally substituted alkyl, heteroalkyl, cycloalkyl, heterocyclic, aryl, heteroaryl or acyl moiety; wherein Rₐ is as defined in subset lxvi) below;
ii. at least one occurrence of R¹ is hydrogen;
iii. at least one occurrence of R¹ is —NO₂;
iv. at least one occurrence of R¹ is —NH₂;
v. at least one occurrence of R¹ is —COOH, —C(═O)OCH₃, —COCH₃, —CONH₂, —SO₂OH, —SO₂CH₃, —SO₂CF₃, —OPO₂OH, —NHC(═O)CH₃, —NHC(═O)CF₃, —NHC(═O)CH₃, —NHC(═O)CF₃, —NHSO₂CH₃ or —NHSO₂CF₃.
vi. at least one occurrence of R¹ is halogen;
vii. at least one occurrence of R¹ is an optionally substituted N-linked heterocyclic group;
viii. at least one occurrence of R¹ is an optionally substituted N-pyrrolyl group;
ix. at least one occurrence of R¹ is an aliphatic moiety;
x. at least one occurrence of R¹ is an alkyl moiety;
xi. at least one occurrence of R¹ is a lower alkyl moiety;
xii. m is 1 and at least one occurrence of R¹ is ortho to the bond to the phthalazinone ring;
xiii. m is 1 and at least one occurrence of R¹ is meta to the bond to the phthalazinone ring;
xiv. each occurrence of R¹ is independently hydrogen, —NO₂, —NH₂, —COOH, —C(═O)OCH₃, —COCH₃, —CONH₂, —SO₂OH, —SO₂CH₃, —SO₂CF₃, —OPO₂OH, —NHC(═O)CH₃, —NHC(═O)CF₃, —NHSO₂CH₃, —NHSO₂CF₃, halogen, an optionally substituted N-linked heterocyclic group or an aliphatic moiety;
xv. each occurrence of R¹ is independently hydrogen, —NO₂, —NH₂, —COOH, —C(═O)OCH₃, —COCH₃, —CONH₂, —SO₂OH, —SO₂CH₃, —SO₂CF₃, —OPO₂OH, —NHC(═O)CH₃, —NHC(═O)CF₃, —NHSO₂CH₃, —NHSO₂CF₃, halogen, an optionally substituted N-pyrrolyl group or a lower alkyl moiety;
xvi. R² and R³ are independently hydrogen, hydroxyl, —NH₂, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a} or —C(═O)R^{c}; wherein n is 0-2, R^{R} is an optionally substituted alkyl, heteroalkyl, cycloalkyl, heterocyclic, aryl, heteroaryl or acyl moiety; or R² and R³ taken together with the nitrogen to which they are attached form a optionally substituted heteroaryl or heterocyclic group comprising 4-10 ring members and 0-3 additional heteroatoms selected from the group consisting of O, N and S; the heteroaryl or heterocyclic group optionally further substituted with one or more optionally substituted alkyl, cycloalkyl, heteroalkyl, heterocyclic, aryl, heteroaryl or acyl groups; wherein Rₐ is as defined in subset lxvi) below;
xvii. R² and R³ are independently hydrogen, hydroxyl, —NH₂, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a} or —C(═O)OR^{a}; wherein n is 0-2, R^{R} is an optionally substituted alkyl, heteroalkyl, cycloalkyl, heterocyclic, aryl, heteroaryl or acyl moiety; wherein Rₐ is as defined in subset lxvi) below;
xviii. R² and R³ are independently hydrogen, lower alkyl or aryl;
xix. R² and R³ are independently hydrogen or lower alkyl;
xx. R² and R³ are independently a hydrophobic group;
xxi. R² and R³ are independently an aliphatic group;
xxii. R² and R³ are independently an unsubstituted aliphatic group;
xxiii. R² and R³ are independently a cyclic or acyclic C₆₋₁₂alkyl, C₆₋₁₂alkenyl, or C₆₋₁₂alkynyl group;
xxiv. R² and R³ are independently an unsubstituted cyclic or acyclic C₆₋₁₂alkyl, C₆₋₁₂alkenyl, or C₆₋₁₂alkynyl group;
xxv. R² and R³ are independently is an -(alkyl)aryl group;
xxvi. R² and R³ are independently a unsubstituted -(alkyl)aryl group;
xxvii. R² and R³ taken together with the nitrogen to which they are attached form a optionally substituted heteroaryl or heterocyclic group comprising 4-10 ring members and 0-3 additional heteroatoms selected from the group consisting of O, N and S; the heteroaryl or heterocyclic group optionally further substituted with one or more optionally substituted alkyl, cycloalkyl, heteroalkyl, heterocyclic, aryl, heteroaryl or acyl groups;
xxviii. R² and R³ taken together with the nitrogen atom to which they are attached form an optionally substituted pyrrolyl, pyrrolidinyl, imidazolyl, imidazolidinyl, pyrazolyl, pyrazolidinyl, 1,2,3-triazolyl, piperidinyl, morpholinyl, thiomorpholinyl, piperazinyl, indolyl, isoindolyl, indolinyl, indazolyl, benzimidazolyl or purinyl moiety;
xxix. R² and R³ taken together with the nitrogen to which they are attached form an optionally substituted 6-membered heterocyclic group comprising 0-3 additional heteroatoms selected from the group consisting of O, N and S;
xxx. R² and R³, taken together, represent the hydrophobic portion of an optionally substituted N-linked ring;
xxxi. R² and R³, taken together, represent the hydrophobic portion of an N-linked ring substituted with hydrophobic groups, such as one or more aliphatic groups;
xxxii. R² and R³, taken together, represent the hydrophobic portion of an optionally substituted piperidinyl ring;
xxxiii. R² and R³, taken together, represent the hydrophobic portion of a piperidinyl ring substituted with hydrophobic groups, such as one or more aliphatic groups;
xxxiv. each occurrence of R⁴ is independently hydrogen, halogen, hydroxyl, —NO₂, —NH₂, —CN, an optionally substituted alkyl, heteroalkyl, cycloalkyl, heterocyclic, aryl or heteroaryl moiety, —OR^{R}, —S(═O)ₙR^{d}, —NR^{b}R^{c}, —C(═O)R^{a}, —OPO₂OR^{a} or —C(═O)OR^{a}; wherein n is 0-2, R^{R} is an optionally substituted alkyl, heteroalkyl, cycloalkyl, heterocyclic, aryl, heteroaryl or acyl moiety; wherein Rₐ, R_{b}, R_{c} and R_{d} are as defined in subsets lxvi), lxvii) and lxviii) below;
xxxv. at least one occurrence of R⁴ is hydrogen;
xxxvi. at least one occurrence of R⁴ is a hydrophobic group;
xxxvii. at least one occurrence of R⁴ is an optionally substituted aliphatic group;
xxxviii. at least one occurrence of R⁴ is an unsubstituted aliphatic group;
xxxix. at least one occurrence of R⁴ is an optionally substituted cyclic or acyclic C₆₋₁₂alkyl, C₆₋₁₂alkenyl, or C₆₋₁₂alkynyl group;
xl. at least one occurrence of R⁴ is an unsubstituted cyclic or acyclic C₆₋₁₂alkyl, C₆₋₁₂alkenyl, or C₆₋₁₂alkynyl group;
xli. at least one occurrence of R⁴ is an optionally substituted -(alkyl)aryl group;
xlii. at least one occurrence of R⁴ is a unsubstituted -(alkyl)aryl group;
xliii. at least one occurrence of R⁴ is —NR^{b}R^{c};
xliv. at least one occurrence of R⁴ is —NH₂;
xlv. at least one occurrence of R⁴ is —C(═O)OR^{a}; wherein Rₐ is as defined in subset lxvi) below;
xlvi. at least one occurrence of R⁴ is —CO₂H;
xlvii. p is ≧3 and each occurrence of R⁴ is independently a cyclic or acyclic C₁₋₆alkyl, C₂₋₆alkenyl, C₂₋₆alkynyl or —(C₁₋₆alkyl)aryl group;
xlviii. p is ≧3 and each occurrence of R⁴ is independently methyl, ethyl, propyl, butyl, pentyl, hexyl, i-propyl or benzyl;
xlix. each occurrence of R⁴ is independently hydrogen, halogen, an optionally substituted aliphatic group, —NR^{b}R^{c}, or —C(═O)OR^{a}, wherein Rₐ, R_{b} and R_{c} are as defined in subsets lxvi) and lxvii) below;
l. each occurrence of R⁴ is independently hydrogen, halogen, an optionally substituted cyclic or acyclic C₆₋₁₂alkyl, C₆₋₁₂alkenyl, or C₆₋₁₂alkynyl group, an optionally substituted -(alkyl)aryl group, —NH₂ or —CO₂H;
li. m is 0;
lii. m is 1;
liii. m is 2;
liv. m is 3;
lv. m is 4;
lvi. p is 0;
lvii. p is 1;
lviii. p is 2;
lix. p is 3;
lx. p is 4;
lxi. p is 5;
lxii. p is 6;
lxiii. q is 1;
lxiv. q is 2;
lxv. q is 4;
lxvi. Ra, for each occurrence, is independently hydrogen or an optionally substituted alkyl, heteroalkyl, cycloalkyl, heterocyclic, aryl or heteroaryl moiety;
lxvii. Rb and Rc, for each occurrence, are independently hydrogen, hydroxy, SO2Rd, or an alkyl, heteroalkyl, cycloalkyl, heterocyclic, aryl, heteroaryl or acyl moiety;
lxviii. Rd, for each occurrence, is independently hydrogen, —N(R^{e})₂, alkyl, heteroalkyl, cycloalkyl, heterocyclic, aryl or heteroaryl;
lxix. Re, for each occurrence, is independently hydrogen or alkyl; and/or
lxx. Cy is one of:

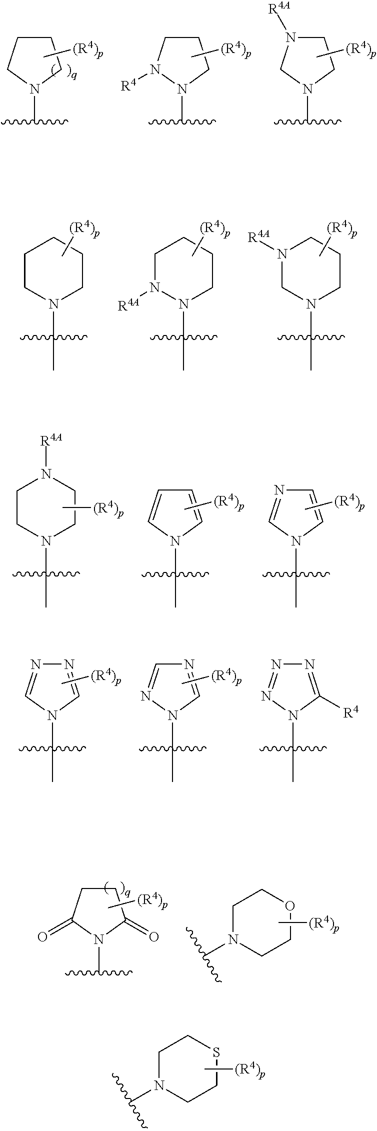

wherein q is 1, 2 or 4 and p and R⁴ are as defined in classes and subclasses herein, and R^{4A} is hydrogen, hydroxy, SO₂R^{d}, or an alkyl, heteroalkyl, cycloalkyl, heterocyclic, aryl, heteroaryl or acyl moiety; wherein R^{d} is as defined in classes and subclasses herein.

It will be appreciated that for each of the classes and subclasses described above and herein, any one or more occurrences of groups such as aliphatic, heteroaliphatic, alkyl, heteroalkyl may independently be substituted or unsubstituted, linear or branched, saturated or unsaturated; and any one or more occurrences of alicyclic, heterocyclic, cycloalkyl, aryl, heteroaryl, cycloaliphatic, cycloheteroaliphatic may be substituted or unsubstituted.

The reader will also appreciate that all possible combinations of the variables described in i)-through lxx) above (e.g., R¹-R⁴, m, p and q, among others) are considered part of the invention. Thus, the invention encompasses any and all compounds of formula (XV), (XVI), (XVII) and (XVIII), and subclasses thereof, generated by taking any possible permutation of variables R¹-R⁴, m, p and q, and other variables/substituents (e.g., R_{a-c}, etc.) as further defined for R¹R⁴, described in i)-through lxx) above, leading to a stable compound.

As the reader will appreciate, compounds of particular interest for the uses herein include, among others, those which share the attributes of one or more of the foregoing subclasses. Some of those subclasses are illustrated by the following sorts of compounds:

Compounds of the formula (and pharmaceutically acceptable derivatives thereof):

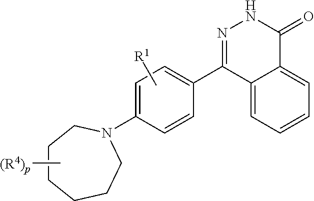

wherein p, R¹ and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

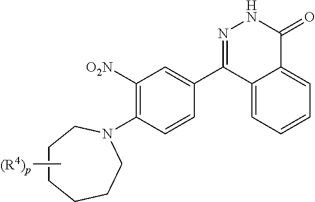

wherein p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

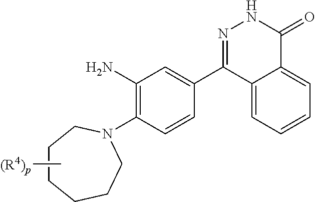

wherein p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

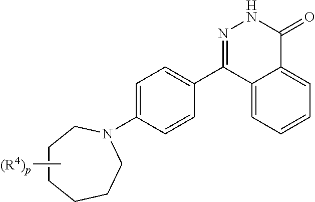

wherein p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

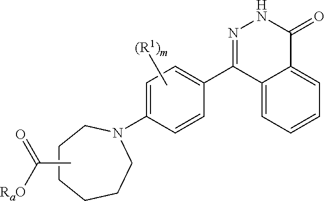

wherein m, R¹ and Rₐ are as defined in classes and subclasses herein. In certain embodiments, Rₐ is hydrogen. In certain embodiments, Rₐ is lower alkyl. In certain embodiments, Rₐ is a hydrophilic group. In certain embodiments, Rₐ is an optionally substituted cyclic or acyclic C₆₋₁₂alkyl, C₆₋₁₂alkenyl, or C₆₋₁₂alkynyl group. In certain embodiments, Rₐ is an optionally substituted -(alkyl)aryl group.

Compounds of the formula (and pharmaceutically acceptable derivatives thereof):

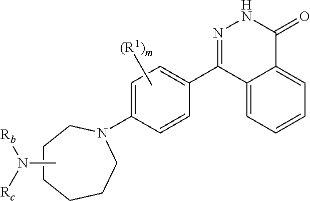

wherein m, R¹, R_{b} and R_{c} are as defined in classes and subclasses herein. In certain embodiments, R_{b} and R_{c} are independently hydrogen or lower alkyl. In certain embodiments, R_{b} and R_{c} are independently a hydrophilic group. In certain embodiments, R_{b} and R_{c} are independently an optionally substituted cyclic or acyclic C₆₋₁₂alkyl, C₆₋₁₂alkenyl, or C₆₋₁₂alkynyl group. In certain embodiments, R_{b} and R_{c} are independently an optionally substituted -(alkyl)aryl group.

Compounds of the formula (and pharmaceutically acceptable derivatives thereof):

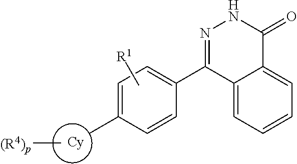

wherein Cy, p, R¹ and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

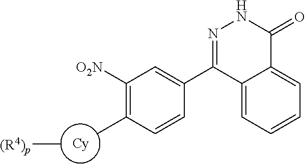

wherein Cy, p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

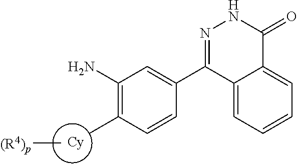

wherein Cy, p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

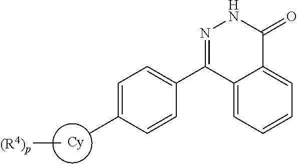

wherein Cy, p and R⁴ are as defined in classes and subclasses herein. In certain embodiments, p is 1-4 and each occurrence of R⁴ is independently hydrogen or lower alkyl. In certain embodiments, at least one occurrence of R⁴ is a hydrophilic group.

Compounds of the formula (and pharmaceutically acceptable derivatives thereof):

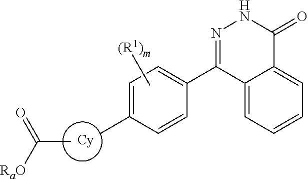

wherein Cy, m, R¹ and Rₐ are as defined in classes and subclasses herein. In certain embodiments, Rₐ is hydrogen. In certain embodiments, Rₐ is lower alkyl.

Compounds of the formula (and pharmaceutically acceptable derivatives thereof):

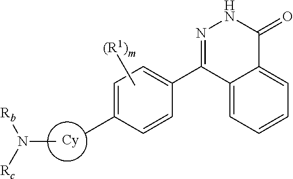

wherein Cy, m, R¹, R_{b} and R_{c} are as defined in classes and subclasses herein. In certain embodiments, R_{b} and R_{c} are independently hydrogen or lower alkyl.

In certain embodiments, for compounds of the classes above, at least one occurrence of R⁴ is a hydrophobic group. In certain embodiments, each occurrence of R⁴ is independently a hydrophobic group. In certain embodiments, the hydrophobic group is an aliphatic group. In certain embodiments, the hydrophobic group is an unsubstituted aliphatic group. In certain embodiments, the hydrophobic group is a cyclic or acyclic C₆₋₁₂alkyl, C₆₋₁₂alkenyl, or C₆₋₁₂alkynyl group. In certain embodiments, the hydrophobic group is an unsubstituted cyclic or acyclic C₆₋₁₂alkyl, C₆₋₁₂alkenyl, or C₆₋₁₂alkynyl group. In certain embodiments, the hydrophobic group is a -(alkyl)aryl group. In certain embodiments, the hydrophobic group is an unsubstituted -(alkyl)aryl group.

In certain embodiments, for compounds of the classes above, m is 0-2. In certain embodiments, m is 0. In certain embodiments, m is 1.

In certain embodiments, for compounds of the classes above, p is 0-2. In certain embodiments, p is 0. In certain embodiments, p is 1.

Non-limiting examples of compounds of the invention in Formula (XV) include:

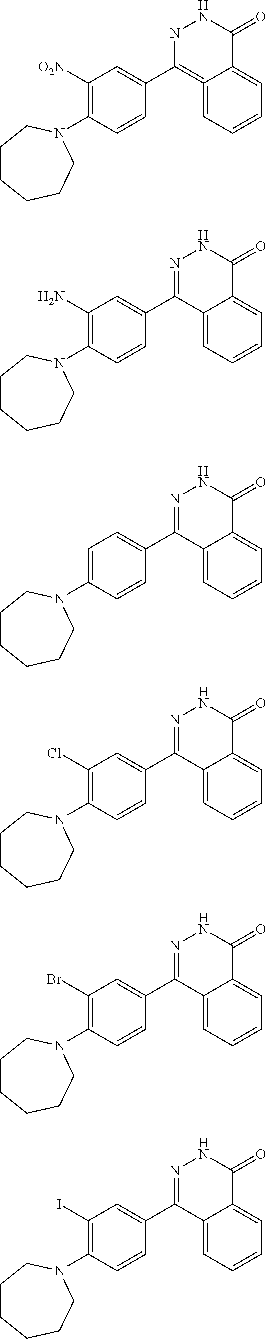

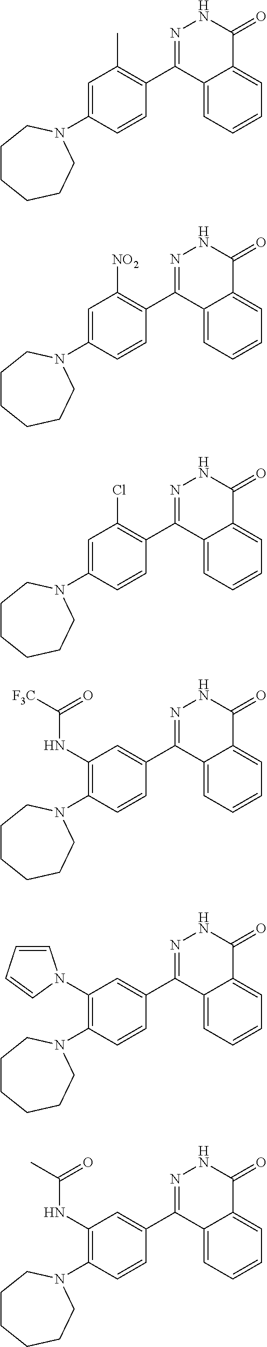

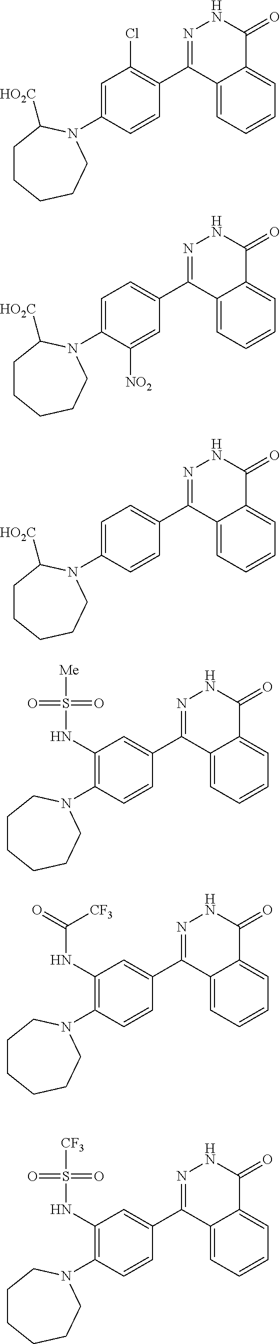

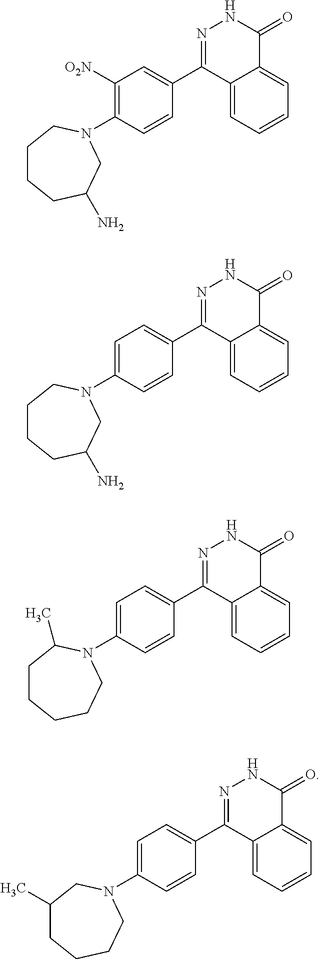

Examples of compounds of Formula (XVI) where R² and R³ do not form a ring include:

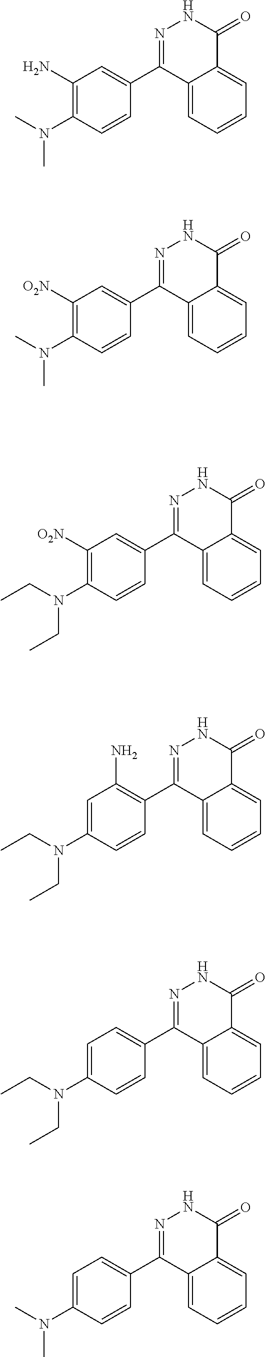

Non-limiting examples of compounds of Formula (XVI) wherein the —NR²R³ moiety forms a ring, optionally further substituted, include the following compounds:

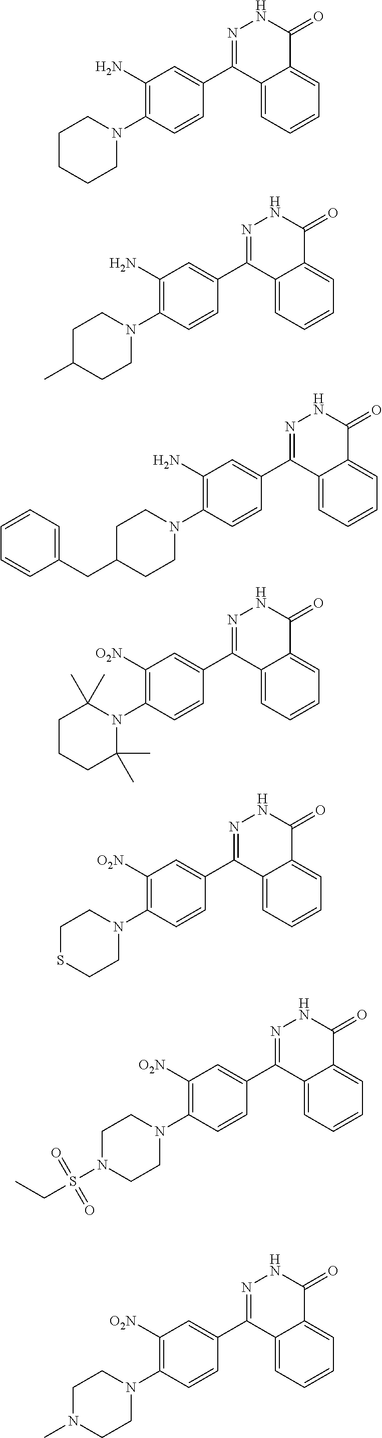

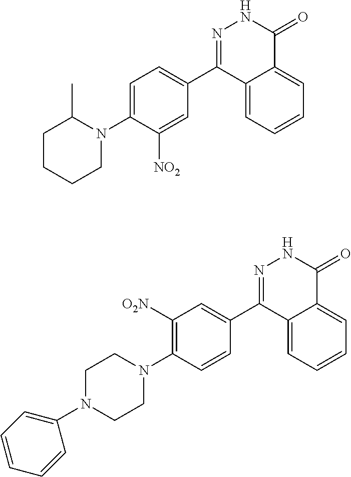

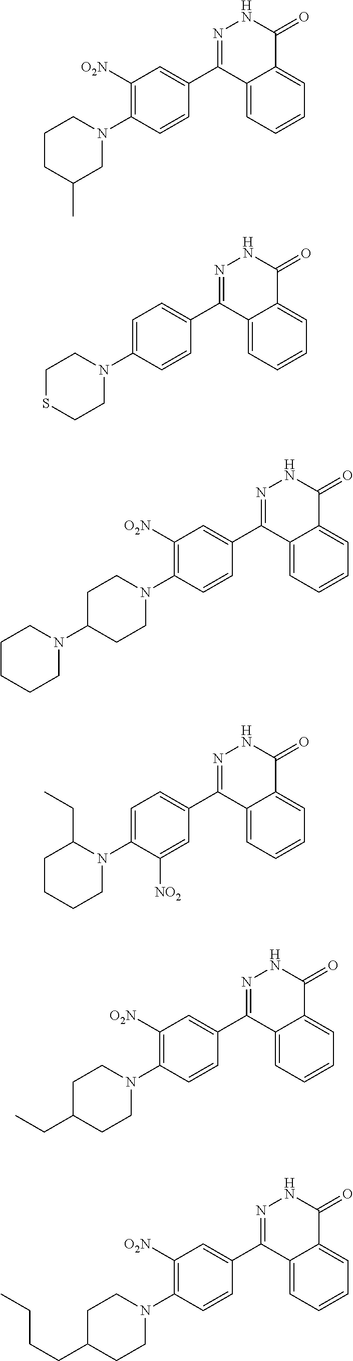

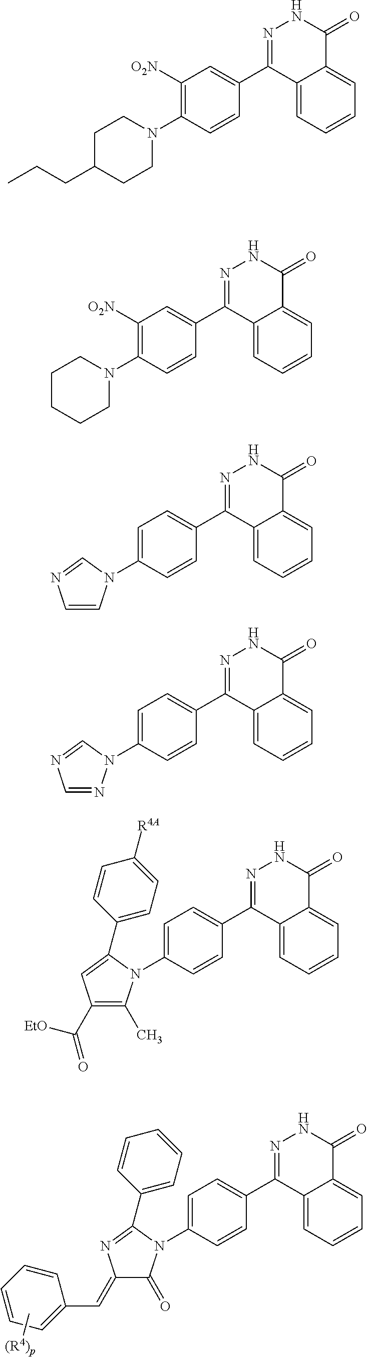

wherein R^{4A} is hydrogen, methyl, methoxy, chloro or —NO₂
wherein p and R⁴ are as defined in classes and subclasses herein.

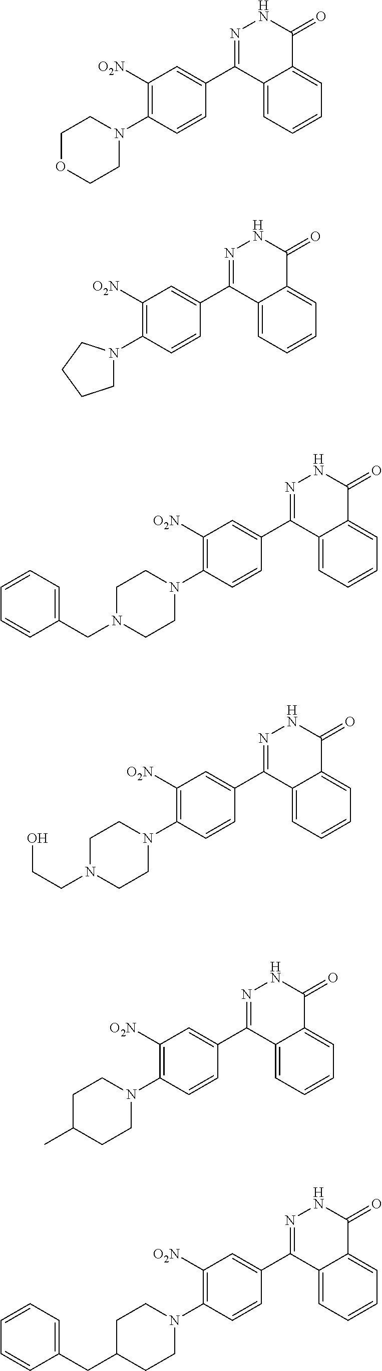

Some of the foregoing compounds from (I) to (XVIII) can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of compound of formula (I)-(XVIII) under different conditions and may exist as one or a combination of polymorphs of compound of general formula (I)-(XVIII) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. In some embodiments, the present invention provides an amorphous compound of formula (I)-(XVIII). Preparation of amorphous solid forms are known in the art and include lyophilization and spray drying. Thus, the present invention encompasses inventive compounds, their derivatives, their tautomeric forms, their stereoisomers, their C(5)-positional isomer their polymorphs, their pharmaceutically acceptable salts their pharmaceutically acceptable solvates and pharmaceutically acceptable compositions containing them. Tautomeric forms of compounds of the present invention include, for example the 3- and 5-substituted pyrazole tautomers of any of the aforementioned disubstituted compounds of general Formula II and related formulas. Likewise, C(5)-positional isomers of the 1,3-disubstituted pyrazoles of general Formula I and III and related formulas are encompassed within the scope of the present invention. Thus, the invention encompasses 1,5-disubstituted pyrazoles.

2) Pharmaceutical Compositions

As discussed above this invention provides methods of use of compounds for enhancing cellular transplantation. Such compounds are described in U.S. Pat. Nos. 7,192,976; 7,250,437; and 7,265,112, in U.S. Patent Application Publication No. 20060116365, and in PCT/US2009/006515, published as WO2010/068287, all of which are incorporated herein by reference in their entireties. Such compounds can be exposed to cells, or to cell donors or cell recipients as compounds embodied herein or as pharmaceutical compositions comprising one or more of said compounds. For exposing to cells, compounds may be provided in compatible solutions containing buffers and other components whose presence and concentrations therein are conducive to maintaining cell viability, such as salts, sugars, plasma or serum or their components, hormones, vitamins, etc. For oral or parenteral administration to a subject donor or recipient, or infusing into an isolated organ or tissue prior to cell harvest, various pharmaceutical compositions are embodied herein.

Accordingly, in one embodiment, the compounds described herein can be used in cellular culture medium, harvesting medium storage medium, or any solutions used that are in contact with the cells during any part of the transplantation process. By way of non-limiting examples of such a solution, the compounds of the invention can be added to the same type of solutions used to maintain the viability of organs and tissues harvested and transported for transplant, such as currently optimally maintained by bathing and transport in storage solutions such as the University of Wisconsin (UW) cold storage solution (100 mM KH₂PO₄, 5 mM MgSO₄ 100 mM potassium lactobionate, 1 mM allopurinol, 3 mM glutathione, 5 mM adenosine, 30 mM raffinose, 50 g/liter of hydroxyethyl starch, 40 units/liter of insulin, 16 mg/liter of dexamethasone, 200,000 units/liter of penicillin, pH 7.4; 320-330 mOsM).

In another aspect of the present invention, pharmaceutical compositions and formulations are provided which comprise any one or more of the compounds described herein (or a prodrug, pharmaceutically acceptable salt or other pharmaceutically acceptable derivative thereof), and a pharmaceutically acceptable carrier. In certain embodiments, these compositions optionally further comprise one or more additional therapeutic agents that would enhance the activity of the compounds embodied herein. In certain embodiments such one or more agents are antiapoptotic, angiogenic or antifibrotic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to HGF/SF activity. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. By way of example, N-methylated pro-drugs of the 3(5)-monosubstituted pyrazoles of the invention are embraced herein.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In one embodiment, liquid compositions or liquid formulations comprising compounds of the invention are provided that have increased solubility as compared to compounds of the invention dissolved in aqueous buffer such as phosphate-buffered saline. In one embodiment, such liquid compositions with increased solubility are provided by a composition comprising polyethylene glycol, polysorbate or a combination thereof. In one embodiment, the polyethylene glycol is polyethylene glycol 300. In another embodiment the polysorbate is polysorbate 80. In another embodiment the polyethylene glycol is present at about 40% to about 60% (v/v). In another embodiment the polysorbate is present at about 5% to about 15% (v/v). In another embodiment the polyethylene glycol is present at about 50% (v/v). In another embodiment the polysorbate is present at about 10% (v/v). In one formulation, the polyethylene glycol is present at 50% (v/v) together with polysorbate 80 at 10% (v/v). The balance of the solution can be a saline solution, a buffer or a buffered saline solution, such as phosphate-buffered saline. The pH of the solution can be from about pH 5 to about pH 9, and in other embodiments, about from pH 6 to about pH 8. In one embodiment the pH of the buffer is 7.4. In the foregoing embodiments, the compound of the invention is soluble at a concentration higher than in buffer alone, and can be present at about 0.8 to about 10 milligrams per milliliter of solution, or even higher. These formulations offer the preparation of convenient dosing solutions of practical volumes for single dose administration, by any route, in particular a parenteral route. In one embodiment, the route is intravenous, subcutaneous or intraperitoneal. Such compositions with a higher solubility permit achievement of more elevated blood concentrations that provide efficacy when the a threshold Cmax (maximal blood concentration after administration) should be achieved for optimal efficacy. Such solutions can also be used as culture medium, storage solution, or other medium to expose cells for transplantation to a compound of the invention.

In certain embodiments, the compounds in the aforementioned cellular culture medium, compositions and formulations useful for purposes described herein include (E)-3(5)-[2-(2,3-methylenedioxyphenyl)vinyl]-1H-pyrazole, (Z)-3(5)-[2-(2,3-methylenedioxyphenyl)vinyl]-1H-pyrazole, (E)-3(5)-[2-(2-chloro-5-trifluoromethylphenyl)vinyl]-1H-pyrazole, (Z)-3 (5)-[2-(2-chloro-5-trifluoromethylphenyl)vinyl]-1H-pyrazole, (E)-3 (5)-[2-(2-trifluoromethylphenyl)vinyl]-1H-pyrazole, (Z)-3(5)-[2-(2-trifluoromethylphenyl)vinyl]-1H-pyrazole, (E)-3(5)-[2-(2-furyl)vinyl]-1H-pyrazole, (Z)-3 (5)-[2-(2-furyl)vinyl]-1H-pyrazole, (E)-3(5)-[2-(2-thienyl)vinyl]-1H-pyrazole, (Z)-3(5)-[2-(2-thienyl)vinyl]-1H-pyrazole, (E)-3-(2-(5-nitrofuran-2-yl)vinyl)-1H-pyrazole, (Z)-3-(2-(5-nitrofuran-2-yl)vinyl)-1H-pyrazole, (E)-3-styryl-1H-pyrazole, (Z)-3-styryl-1H-pyrazole, (E)-2-(2-(1H-pyrazol-3-yl)vinyl)-1H-indole, (Z)-2-(2-(1H-pyrazol-3-yl)vinyl)-1H-indole, (E)-4-(2-(1H-pyrazol-3-yl)vinyl)-N,N-dimethylaniline, (Z)-4-(2-(1H-pyrazol-3-yl)vinyl)-N,N-dimethylaniline, (E)-3-(4-methoxystyryl)-1H-pyrazole, (Z)-3-(4-methoxystyryl)-1H-pyrazole, (E)-3-(2,6-dichlorostyryl)-1H-pyrazole, (Z)-3-(2,6-dichlorostyryl)-1H-pyrazole, (E)-3-(2-(naphthalen-2-yl)vinyl)-1H-pyrazole, (Z)-3-(2-(naphthalen-2-yl)vinyl)-1H-pyrazole, (E)-3-(2-(1H-pyrrol-2-yl)vinyl)-1H-pyrazole, (Z)-3-(2-(1H-pyrrol-2-yl)vinyl)-1H-pyrazole, (E)-3-(2-(thiophen-3-yl)vinyl)-1H-pyrazole, (Z)-3-(2-(thiophen-3-yl)vinyl)-1H-pyrazole, (E)-3-(2-(1H-pyrrol-3-yl)vinyl)-1H-pyrazole, (Z)-3-(2-(1H-pyrrol-3-yl)vinyl)-1H-pyrazole, (E)-3-(2-(furan-3-yl)vinyl)-1H-pyrazole, and (Z)-3-(2-(furan-3-yl)vinyl)-1H-pyrazole. These are merely exemplary and non-limiting. Other exemplary compounds are described in U.S. Pat. No. 6,610,726. Still others are described in U.S. Patent Application Publication No. 20060116365.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the polyethylene glycol-polysorbate formulation described above is useful for injectable administration.

Such injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tabulating lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Formulations for intraocular administration are also included. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects). In non-limiting examples, one or more compounds of the invention may be formulated with at least one cytokine, growth factor or other biological, such as an interferon, e.g., alpha interferon, or with at least another small molecule compound. Non-limiting examples of pharmaceutical agents that may be combined therapeutically with compounds of the invention include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors; antiapoptotic agents; and motility and morphogenic agents.

In certain embodiments, the pharmaceutical compositions of the present invention further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

Pharmaceutical Uses and Methods of Treatment

As discussed above, compositions and formulations comprising compounds as described herein exhibit activity to enhance the cellular transplantation process.

In certain embodiments, the method involves the administration of a therapeutically effective amount of the compound or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

In other embodiments, one or more compounds of the invention are exposed to the cells in vitro or ex vivo, during one or more of the steps from harvest or culture to administration or implantation into the patient. In such cases, compounds can be included in cell culture medium, storage solution, or any other medium in which such cells are contained during any one or more steps. Examples of such solutions are described above, and means for increasing the concentration of inventive compounds in such solutions is also described above. Appropriate concentrations of compound in the various solutions can be readily determined by the skilled artisan. In non-limiting embodiments, the concentration of compound can be from about 0.001 to about 1000 micromolar.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments, the compounds of the invention may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Moreover, pharmaceutical compositions comprising one or more compounds of the invention may also contain other compounds or agents for which co-administration with the compound(s) of the invention is therapeutically advantageous. As many pharmaceutical agents are used in the treatment of the diseases and disorders for which the compounds of the invention are also beneficial, any may be formulated together for administration. Synergistic formulations are also embraced herein, where the combination of at least one compound of the invention and at least one other compounds act more beneficially than when each is given alone. Non-limiting examples of pharmaceutical agents that may be combined therapeutically with compounds of the invention include (non-limiting examples of diseases or conditions treated with such combination are indicated in parentheses): antivirals and antifibrotics, such as interferon alpha (hepatitis B, and hepatitis C), combination of interferon alpha and ribavirin (hepatitis C), Lamivudine (hepatitis B), Adefovir dipivoxil (hepatitis B), interferon gamma (idiopathic pulmonary fibrosis, liver fibrosis, and fibrosis in other organs); anticoagulants, e.g., heparin and warfarin (ischemic stroke); antiplatelets e.g., aspirin, ticlopidine and clopidogrel (ischemic stroke); other growth factors involved in regeneration, e.g., VEGF and FGF and mimetics of these growth factors; antiapoptotic agents; and motility and morphogenic agents.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the preparing solutions or include ready-made solutions for in vitro or ex vivo handling of cells for transplantation. Other kits comprise one or more compounds of the invention to readily add to an existing cell handling solution. Other such kits are especially suited for the delivery of solid oral forms such as tablets or capsules for patients having received a cellular transplant. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

### General Description of Synthetic Methods:

The practitioner has a well-established literature of small molecule chemistry to draw upon, in combination with the information contained herein, for guidance on synthetic strategies, protecting groups, and other materials and methods useful for the synthesis of the compounds of this invention. Guidance can be found in U.S. Pat. Nos. 7,192,976; 7,250,437; and 7,265,112, and in U.S. Patent Application Publication No. 20060116365 and PCT publications WO2006/036981 and WO2010/068287, all of which are incorporated herein by reference in their entireties.

### Biological Activity:

Cryopreserved primary human hepatocytes (CellzDirect catalog #HMCPMS, Lot # Hu0948) were plated on a collagen-coated 96 well plate according to the supplier's instructions and allowed to adhere and stabilize for 48 hours. Cells were treated with a compound of formula (II) embodied herein (10 μM final concentration) or HGF (50 ng/ml) overnight, and the next day, cell proliferation was determined using a BrdU assay (Chemicon, catalog #2750) according to the manufacturer's instructions, with a 4 hour incubation period of BrdU. Plates were read at 450 and 550 (background) nm and the difference was taken as an indicator of cell proliferation. Both test compound and HGF stimulated the BrdU incorporation in hepatocytes vs. control: test compound, 0.336±0.072; HGF, 0.388±0.134; and control, 0.032±0.011. Hepatocytes enhanced using the compounds of the invention can be used subsequently for cellular transplant into a human recipient, providing enhanced cell survival and numbers to increase the quality of the cells used in the transplant, the survival of cells after transplant and reduce the number of cells required to achieve the desired outcome of the transplant procedure and restoring or enhancing hepatic function in the recipient.

Other compounds embodied here can be used for the same purposes on hepatic cells, or on any of numerous other cell types used for cellular transplantation, to increase survival and quality of cells during the transplant procedure, from harvest to implantation. Compounds of the invention can also be administered to the cell donor or transplant recipient, or both, to further enhance the quality and quantity of cells being transplanted during the procedure.

What is claimed is:

1. A method for enhancing cellular transplantation comprising exposing cells intended for transplant, or the donor or recipient thereof, a tissue or organ thereof, or any combination thereof, to a compound or a pharmaceutical composition comprising a compound that is a small molecule mimetic of hepatocyte growth factor/scatter factor.

2. A method for enhancing cellular transplantation comprising exposing cells intended for transplant, or the donor or recipient thereof, a tissue or organ thereof, or any combination thereof, to a compound or a pharmaceutical composition comprising a compound having the 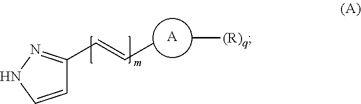
tautomer thereof; or pharmaceutically acceptable derivative thereof;
wherein m is an integer from 1-3 and [C═C]ₘ for each occurrence is independently cis or trans;
A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S;
q is one or more; and
each R is independently selected from the group consisting of hydrogen, halogen, hydroxyl, —NO₂, —CN, an optionally substituted aliphatic, heteroaliphatic, aromatic, heteroaromatic moiety; —OR^{R}, —S(═O)ₙR^{d}, —NR^{b}R^{c}, and —C(═O)R^{a}; wherein n is 0-2, R^{R} is an optionally substituted aliphatic, heteroaliphatic, aromatic, heteroaromatic moiety;
R^{a}, for each occurrence, is independently selected from the group consisting of hydrogen, hydroxy, optionally substituted aliphatic, heteroaliphatic, aryl and heteroaryl;
R^{b} and R^{c}, for each occurrence, are independently selected from the group consisting of hydrogen; hydroxy; SO₂R^{d}; optionally substituted aliphatic, heteroaliphatic, aryl and heteroaryl;
R^{d}, for each occurrence, is independently selected from the group consisting of hydrogen; —N(R^{e})₂; optionally substituted aliphatic, aryl and heteroaryl; and
R^{e}, for each occurrence, is independently hydrogen or optionally substituted aliphatic.

3. The method of claim 1 or 2 wherein the compound is (E)-3(5)-[2-(2,3-methylenedi oxyphenyl)vinyl]-1H-pyrazole, (Z)-3(5)-[2-(2,3-methylenedioxyphenyl)vinyl]-1H-pyrazole, (E)-3(5)-[2-(2-chloro-5-trifluoromethylphenyl)vinyl]-1H-pyrazole, (Z)-3 (5)-[2-(2-chloro-5-trifluoromethyl-phenyl)vinyl]-1H-pyrazole, (E)-3(5)-[2-(2-trifluoromethylphenyl)vinyl]-1H-pyrazole, (Z)-3 (5)-[2-(2-trifluoromethylphenyl)vinyl]-1H-pyrazole, (E)-3 (5)-[2-(2-furyl)vinyl]-1H-pyrazole, (Z)-3(5)-[2-(2-furyl)vinyl]-1H-pyrazole, (E)-3 (5)-[2-(2-thienyl)vinyl]-1H-pyrazole, (Z)-3(5)-[2-(2-thienyl)vinyl]-1H-pyrazole, (E)-3-(2-chloro-4-(trifluoromethyl)styryl)-1H-pyrazole, (Z)-3-(2-chloro-4-(trifluoromethylstyryl)-1H-pyrazole, (E)-3-(4-(diethoxymethyl)styryl)-1H-pyrazole, (Z)-3-(4-(diethoxymethyl)styryl)-1H-pyrazole, (E)-3-(2-(5-nitrofuran-2-yl)vinyl)-1H-pyrazole, (Z)-3-(2-(5-nitrofuran-2-yl)vinyl)-1H-pyrazole, (E)-3-styryl-1H-pyrazole, (Z)-3-styryl-1H-pyrazole, (E)-2-(2-(1H-pyrazol-3-yl)vinyl)-1H-indole, (Z)-2-(2-(1H-pyrazol-3-yl)vinyl)-1H-indole, (E)-4-(2-(1H-pyrazol-3-yl)vinyl)-N,N-dimethylaniline, (Z)-4-(2-(1H-pyrazol-3-yl)vinyl)-N,N-dimethylaniline, (E)-3-(4-methoxystyryl)-1H-pyrazole, (Z)-3-(4-methoxystyryl)-1H-pyrazole, (E)-3-(2,6-dichlorostyryl)-1H-pyrazole, (Z)-3-(2,6-di chloro styryl)-1H-pyrazole, (E)-3-(2-(naphthalen-2-yl)vinyl)-1H-pyrazole, (Z)-3-(2-(naphthalen-2-yl)vinyl)-1H-pyrazole, (E)-3-(2-(1H-pyrrol-2-yl)vinyl)-1H-pyrazole, (Z)-3-(2-(1H-pyrrol-2-yl)vinyl)-1H-pyrazole, (E)-3-(2-(thiophen-3-yl)vinyl)-1H-pyrazole, (Z)-3-(2-(thiophen-3-yl)vinyl)-1H-pyrazole, (E)-3-(2-(1H-pyrrol-3-yl)vinyl)-1H-pyrazole, (Z)-3-(2-(1H-pyrrol-3-yl)vinyl)-1H-pyrazole, (E)-3-(2-(furan-3-yl)vinyl)-1H-pyrazole, or (Z)-3-(2-(furan-3-yl)vinyl)-1H-pyrazole.

4. A method for enhancing cellular transplantation comprising exposing cells intended for transplant, or the donor or recipient thereof, a tissue or organ thereof, or any combination thereof, to a compound or a pharmaceutical composition comprising a compound having the structure of Formula (B): 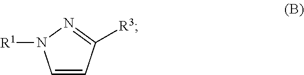
C(5)-positional isomer thereof; or a prodrug, salt, hydrate, or ester thereof;
wherein R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 0-3; AL² is an aliphatic or alicyclic moiety; and AL², the aryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
R³ is a cis or trans CHCHAryl, CHCHHeterocyclic, phenoxyphenyl, or a heterocyclic group, wherein the aryl, heterocyclic or phenoxyphenyl moiety may be optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄alkyl, C₁₋₅ alkoxy, nitro, and N(R^{e})₂; and heteroaryl optionally substituted with one or more substituents independently selected from halogen, hydroxy, C₁₋₄alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆alkyl.

5. The method of claim 1 or 4 wherein the compound is (1-methyl-1H-pyrrol-2-yl)(3-(2-(thiophen-2-yl)vinyl)-1H-pyrazol-1-yl)methanone, (3-(2-(furan-2-yl)vinyl)-1H-pyrazol-1-yl)(1-methyl-1H-pyrrol-2-yl)methanone, (3-(2-(furan-2-yl)vinyl)-1H-pyrazol-1-yl)(6-morpholinopyridin-3-yl)methanone, (3-(2-(furan-2-yl)vinyl)-1H-pyrazol-1-yl)(thiophen-2-yl)methanone, (3-(2-(furan-2-yl)vinyl)-1H-pyrazol-1-yl)(thiophen-3-yl)methanone, (3-(2-(Thiophen-2-yl)vinyl)-1H-pyrazol-1-yl)(thiophen-3-yl)methanone, (5-chloro-4-methoxythiophen-3-yl)(3-(2-(thiophen-2-yl)vinyl)-1H-pyrazol-1-yl)methanone, (5-nitrothiophen-3-yl)(3-(2-(thiophen-2-yl)vinyl)-1H-pyrazol-1-yl)methanone, (6-chloropyridin-3-yl)(3-(2-(thiophen-2-yl)vinyl)-1H-pyrazol-1-yl)methanone, 1-(4-(3-(2-(thiophen-2-yl)vinyl)-1H-pyrazole-1-carbonyl)piperidin-1-yl)ethanone, furan-2-yl(3-(2-(furan-2-yl)vinyl)-1H-pyrazol-1-yl)methanone, furan-2-yl(3-(2-(thiophen-2-yl)vinyl)-1H-pyrazol-1-yl)methanone, thiophen-2-yl(3-(2-(thiophen-2-yl)vinyl)-1H-pyrazol-1-yl)methanone, 3-(5-(3-chlorostyryl)-1H-pyrazol-1-yl)-5-methyl-4H-1,2,4-triazol-4-amine, (3-(2,6-dichlorostyryl)-1H-pyrazol-1-yl)(thiophen-2-yl)methanone, (3-styryl-1H-pyrazol-1-yl)(thiophen-2-yl)methanone, (3-styryl-1H-pyrazol-1-yl)(thiophen-3-yl)methanone, (5-nitrothiophen-3-yl)(3-styryl-1H-pyrazol-1-yl)methanone, (6-morpholinopyridin-3-yl)(3-styryl-1H-pyrazol-1-yl)methanone, or furan-2-yl(3-styryl-1H-pyrazol-1-yl)methanone.

6. A method for enhancing cellular transplantation comprising exposing cells intended for transplant, or the donor or recipient thereof, a tissue or organ thereof, or any combination thereof, to a compound or a pharmaceutical composition comprising a compound having Formula (XV) or (XVI): 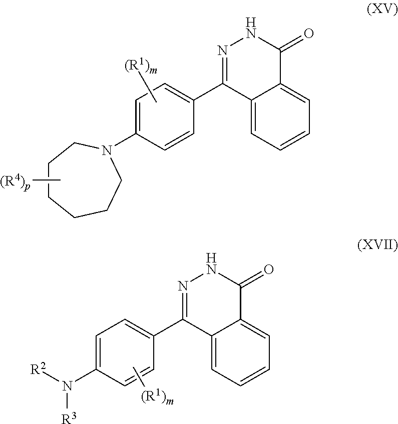
or pharmaceutically acceptable derivatives thereof;
wherein m is an integer from 1 to 4;
p is an integer from 1 to 6;
each occurrence of R¹ and R⁴ is independently hydrogen, halogen, hydroxyl, —NO₂, —NH₂, —CN, an optionally substituted aliphatic, alicyclic, heteroaliphatic, heterocyclic, aromatic or heteroaromatic moiety, —OR^{R}, —S(═O)ₙR^{d}, —NR^{b}R^{c}, —C(═O)R^{a}, —OPO₂OR^{a} or —C(═O)OR^{a}; wherein n is 0-2, R^{R} is an optionally substituted aliphatic, alicyclic, heteroaliphatic, heterocyclic, aromatic, heteroaromatic or acyl moiety;
R² and R³ are independently hydrogen, hydroxyl, —NH₂, an optionally substituted aliphatic, heteroaliphatic, alicyclic, heterocyclic, aromatic or heteroaromatic moiety, —OR^{R}, —S(═O)ₙR^{d}, —NR^{b}R^{c}, —C(═O)R^{a} or —C(═O)OR^{a}; wherein n is 0-2, R^{R} is an optionally substituted aliphatic, heteroaliphatic, alicyclic, heterocyclic, aromatic or heteroaromatic or acyl moiety; or R² and R³ taken together with the nitrogen to which they are attached form an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;
R^{a}, for each occurrence, is independently selected from the group consisting of hydrogen and an optionally substituted aliphatic, alicyclic, heteroaliphatic, heterocyclic, aromatic, or heteroaromatic moiety;
R^{b} and R^{c}, for each occurrence, are independently selected from the group consisting of hydrogen; hydroxy; SO₂R^{d}; and aliphatic, alicyclic, heteroaliphatic, heterocyclic, aromatic, heteroaromatic or acyl moiety;
R^{d}, for each occurrence, is independently selected from the group consisting of hydrogen; —N(R^{e})₂; aliphatic, alicyclic, heteroaliphatic, heterocyclic, aromatic or heteroaromatic; and
R^{e} for each occurrence, is independently hydrogen or aliphatic.

7. The method of claim 1 or 6 wherein the compound is selected from among 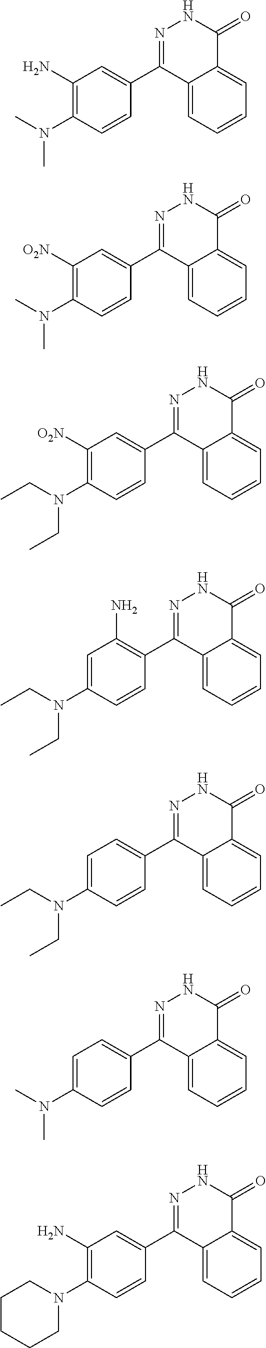 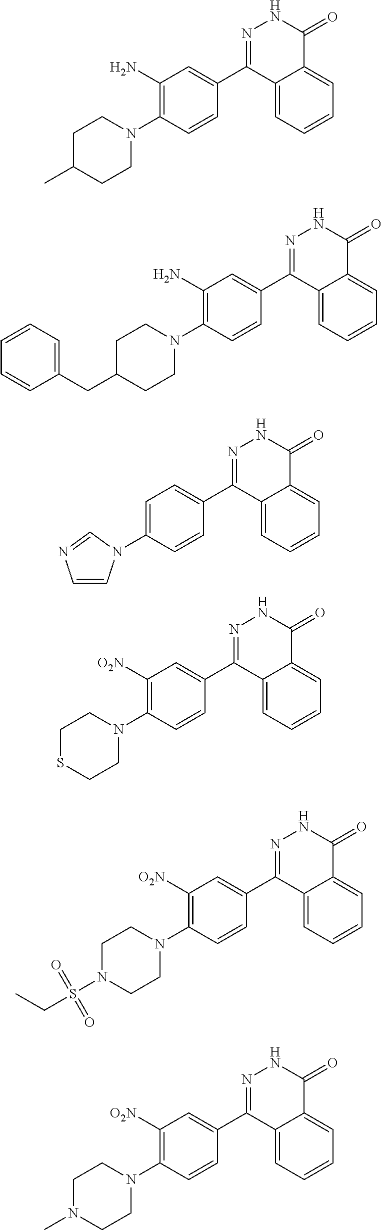 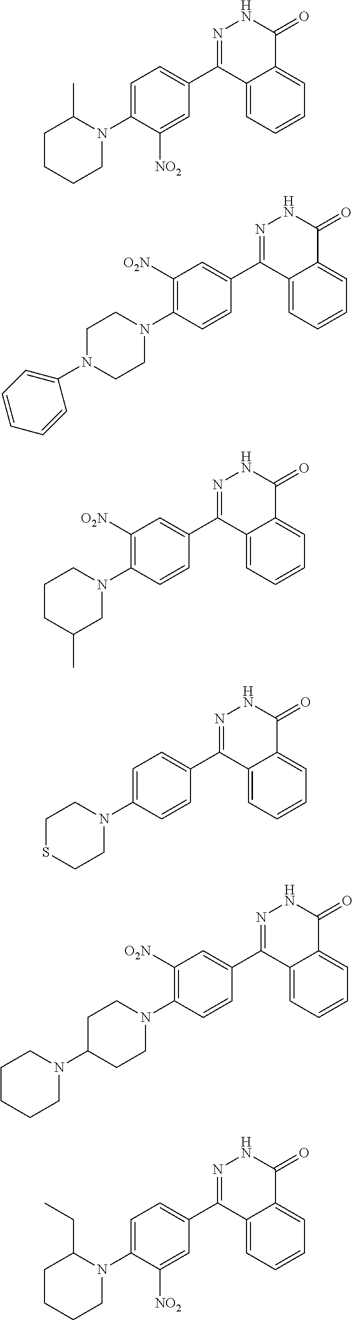 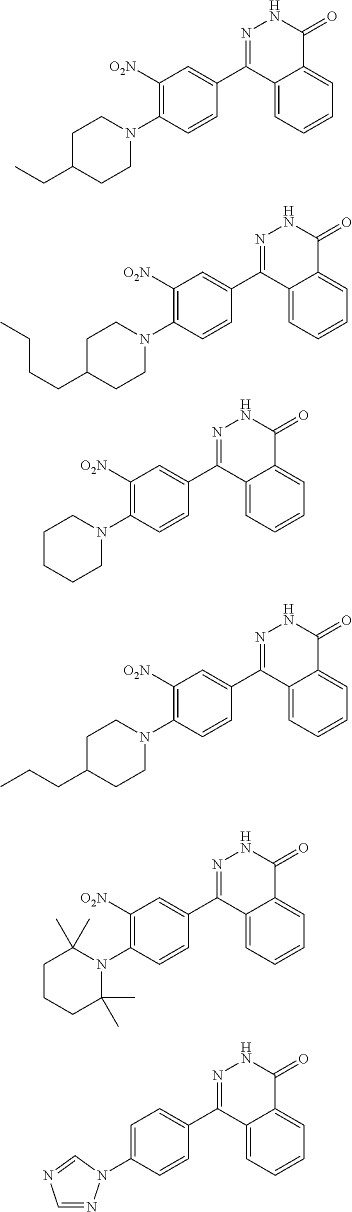 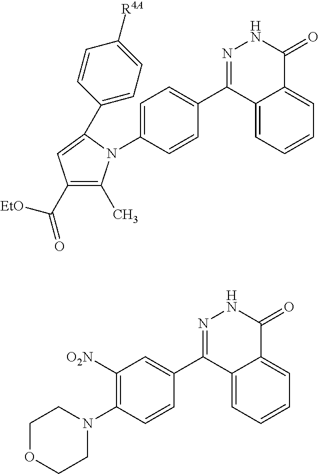
wherein R^{4A} is hydrogen, methyl, methoxy, chloro or —NO₂

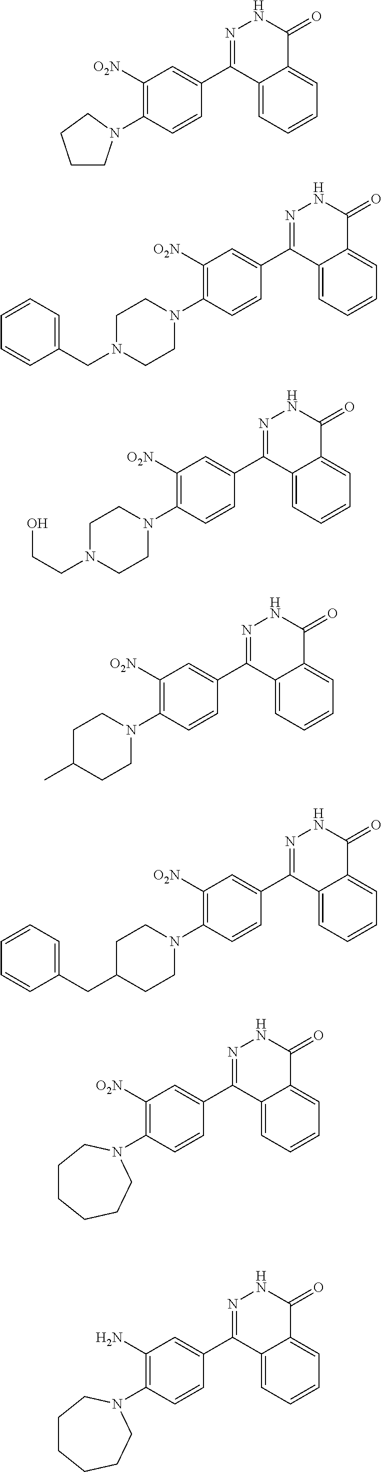

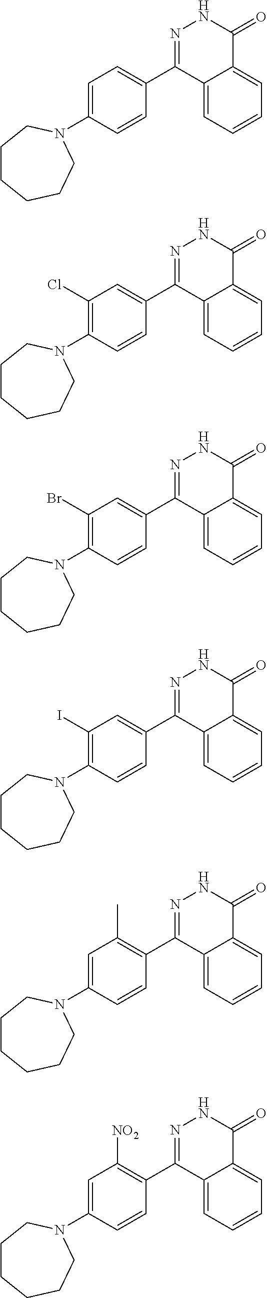

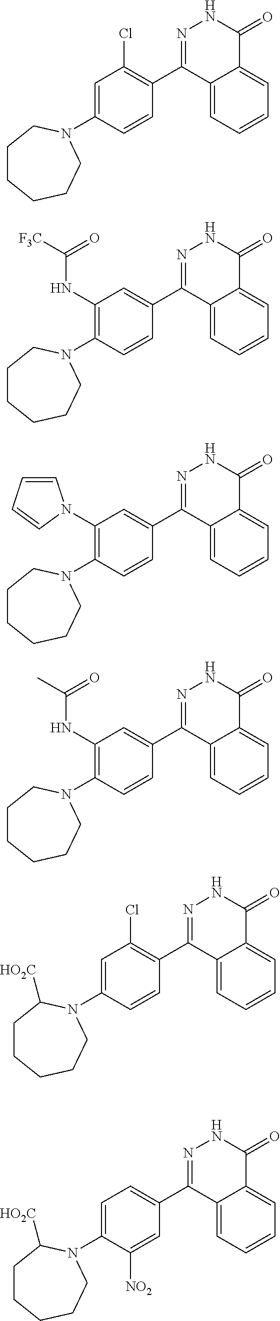

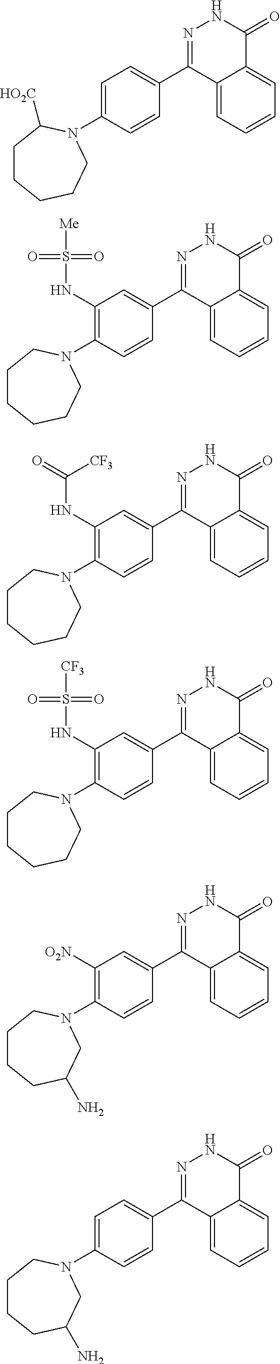

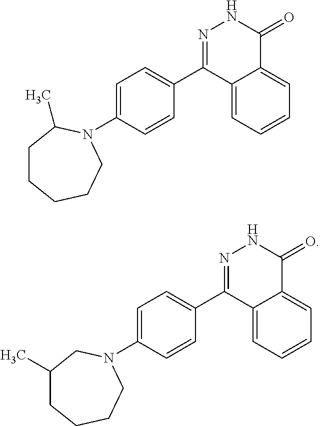

8. The method of any one of claims 1-7 wherein the cells are hepatocytes, islet cells, myocardial cells, stem cells, bone marrow derived cells, leukocytes, myoblasts or neuronal cells.

9. The method of any one of claims 1-8 wherein the compound is administered to the donor before or during harvesting of cells for transplant, or a tissue or organ thereof.

10. The method of any one of claims 1-8 wherein the compound is administered to the recipient before, after, or both before and after the cells are transplanted into said recipient.

11. The method of any one of claims 1-8 wherein the cells are exposed to the compound in vitro or ex vivo.

12. The method of claim 11 wherein the cells are exposed to the compound in primary culture.

13. The method of claim 11 wherein the cells are exposed to the compound during a step comprising harvesting, isolation, purification, culture, storage, transfer, incubation, washing, administration, or any combination of any of the foregoing.

14. The method of claim 1 wherein the cells express c-met.
